SEQUENCE LISTING (23 sequences)

SEQ ID NO: 1 - DNA, 23 bp, Artificial Sequence
  Description of Artificial Sequence Synthetic primer
caaagcatgcctgcaggaattcg

SEQ ID NO: 2 - DNA, 24 bp, Artificial Sequence
  Description of Artificial Sequence Synthetic primer
gagcgcagcgagtcagtgagcgag

SEQ ID NO: 3 - DNA, 21 bp, Artificial Sequence
  Description of Artificial Sequence Synthetic primer
gccaaacctacaggtggggtc

SEQ ID NO: 4 - DNA, 34 bp, Artificial Sequence
  Description of Artificial Sequence Synthetic primer
tttcattataaaacccctcataaaaaccccacag

SEQ ID NO: 5 - DNA, 55 bp, Artificial Sequence
  Description of Artificial Sequence Synthetic primer
ctgtggggtttttatgaggggttttataatgaaagaccccacctgtaggtttggc

SEQ ID NO: 6 - DNA, 56 bp, Artificial Sequence
  Description of Artificial Sequence Synthetic primer
gaagggactcagaccgcagaatctgagtgccccccgagtgaggggttgtgggctct

SEQ ID NO: 7 - DNA, 26 bp, Artificial Sequence
  Description of Artificial Sequence Synthetic primer
agagcccacaacccctcactcggggg

SEQ ID NO: 8 - DNA, 30 bp, Artificial Sequence
  Description of Artificial Sequence Synthetic primer
gcactcagattctgcggtctgagtcccttc

SEQ ID NO: 9 - DNA, 150 bp, Artificial Sequence
  Description of Artificial Sequence Synthetic nucleotide construct
caaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataa

SEQ ID NO: 10 - DNA, 447 bp, Artificial Sequence
  Description of Artificial Sequence Synthetic nucleotide construct
tgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggg

SEQ ID NO: 11 - DNA, 6418 bp, Artificial Sequence
  Description of Artificial Sequence Synthetic plasmid pONY8.1G
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 12 - DNA, 9952 bp, Artificial Sequence
  Description of Artificial Sequence Synthetic plasmid pONY8.4ZCG
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagaactagtggatcccccgggctgcaggaattcgatatcaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaaaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 13 - DNA, 539 bp, Artificial Sequence
  Description of Artificial Sequence Synthetic polynucleotide
tgtggggtttttatgaggggttttataatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaat

SEQ ID NO: 14 - DNA, 9138 bp, Artificial Sequence
  Description of Artificial Sequence Synthetic plasmid pONY8.1Zhyb
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 15 - DNA, 98 bp, Artificial Sequence
  Description of Combined DNA/RNA Molecule Synthetic oligonucleotide
guaaauaaaactgggcttgtcgagacagagaagactcttgcgtttctgataggcacctattggtcttactgacatccactttgcctttctctccacag

SEQ ID NO: 16 - DNA, 114 bp, Artificial Sequence
  Description of Combined DNA/RNA Molecule Synthetic oligonucleotide
agaucauaagguaaauaaaactgggcttgtcgagacagagaagactcttgcgtttctgataggcacctattggtcttactgacatccactttgcctttctctccacaggaugcu

SEQ ID NO: 17 - RNA, 48 nt, Artificial Sequence
  Description of Artificial Sequence Synthetic oligonucleotide
agaucauaaggaugcuaauccucugagagnnnagnnncgaaaugaucu

SEQ ID NO: 18 - DNA, 49 bp, Artificial Sequence
  Description of Artificial Sequence Synthetic oligonucleotide
aaactgggcttgtcgagacagagaagactcttgcgtttctgataggcac

SEQ ID NO: 19 - DNA, 48 bp, Artificial Sequence
  Description of Artificial Sequence Synthetic oligonucleotide
ctattggtcttactgacatccactttgcctttctctccacaggatgct

SEQ ID NO: 20 - DNA, 22 bp, Artificial Sequence
  Description of Artificial Sequence Synthetic oligonucleotide
gggttgcccnnnngggcgaccc

SEQ ID NO: 21 - RNA, 51 nt, Artificial Sequence
  Description of Artificial Sequence Synthetic oligonucleotide
gggcgacccugaugagnnnagnnncgaaacggugaaagccguagguugccc

SEQ ID NO: 22 - DNA, 395 bp, Artificial Sequence
  Description of Artificial Sequence Synthetic nucleotide construct
gggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctc

SEQ ID NO: 23 - DNA, 9924 bp, Artificial Sequence
  Description of Artificial Sequence Synthetic plasmid pONY8.4GCZ
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaaaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

(19) United States
(12) Patent Application Publication
(10) Pub. No.: US 2009/0007284 A1
(43) Pub. Date: Jan. 1, 2009

(54) TRANSGENIC ORGANISM

(76) Inventors: Philippa Radcliffe, Oxford (GB); Kyriacos Mitrophanous, Oxford (GB); Michael Themis, London (GB)

Correspondence Address: FROMMER LAWRENCE & HAUG, 745 FIFTH AVENUE- 10TH FL., NEW YORK, NY, 10151, US

(21) Appl. No.: 12/014,057

(22) Filed: Jan. 14, 2008

Related U.S. Application Data
(63) Continuation of application No. 10/421,947, filed on Apr. 24, 2003, now abandoned.
Application No. 10/421,947 is a continuation-in-part of application No. PCT/GB02/05901, filed on Dec. 23, 2002.
Application No. PCT/GB02/05901 is a continuation-in-part of application No. 10/082,122, filed on Feb. 26, 2002, now abandoned.

(30) Foreign Application Priority Data
Dec. 21, 2001 (GB) 0130797.4
Jan. 18, 2002 (GB) 0201140.1
May 17, 2002 (GB) GB 0211409.8

(51) Int. Cl. A01K 67/027; C12N 15/87; C12N 5/06

(52) U.S. Cl. 800/19; 435/455; 800/25; 435/349

(57) ABSTRACT

A method of producing a transgenic cell comprising introducing into a cell a non-primate lentiviral expression vector comprising a nucleotide of interest (NOI). Also described is a method of producing a transgenic cell comprising introducing into a cell a lentiviral expression vector comprising a NOI capable of generating an antisense oligonucleotide, a ribozyme, an siRNA, a short hairpin RNA, a micro-RNA or a group 1 intron. Also described is a viral vector comprising a first nucleotide sequence, wherein said first nucleotide sequence comprises: (a) a second nucleotide sequence comprising an aptazyme; and (b) a third nucleotide sequence capable of generating a polynucleotide; wherein (a) and (b) are operably linked and wherein the aptazyme is activatable to cleave a transcript of the first nucleotide sequence such that said polynucleotide is generated.

### FIELD OF THE INVENTION

The present invention relates to a method for producing a transgenic cell and a transgenic organism.

### BACKGROUND TO THE INVENTION

The ability to introduce genes and/or other DNA sequences into the germline or somatic cells of organisms such as mammals is one of the greatest technical advances in recent biology. Such animals are said to be transgenic. When germline changes are involved, the results of genetic manipulation are inherited by the offspring of the animals and all cells of these offspring inherit the introduced gene and in some cases deleted DNA as part of their genetic make-up. Transgenic mammals have provided a means of studying gene regulation during embryogenesis and in differentiation, for studying the action of oncogenes, and for studying the intricate interactions of cells in the immune system. The whole animal is the ultimate assay system for manipulating genes which direct complex biological processes. In addition, transgenic animals provide exciting possibilities for expressing useful recombinant proteins and for generating precise animal models of human genetic disorders.

The production of transgenic animals is commonly done in one of two ways: by targeted insertion of DNA by homologous recombination in embryonic stem (ES) cells which is a labour intensive and time-consuming process, or by pronuclear injection of a fertilised ovum in which integration of DNA is random and may lead to an insertion of large tandem arrays of DNA which are unstable and subject to rearrangements and deletions in subsequent cell divisions. WO99/51755 discusses use of a retroviral expression vector comprising a nucleic acid encoding at least one ribozyme for production of a transgenic animal. No specific disclosure is made of the retrovirus used in the specific example. Mention is also made of the possibility of using an adenovirus, an adeno-associated virus, a lentivirus, a herpes simplex virus or a vaccinia virus. However there are no specific examples of the use of these viruses.

Thus, in recent years, retroviruses have been proposed for use in gene therapy. Essentially, retroviruses are RNA viruses with a life cycle different to that of lytic viruses. In this regard, when a retrovirus infects a cell, its genome is converted to a DNA form. In other words, a retrovirus is an infectious entity that replicates through a DNA intermediate. More details on retroviral infection etc. are presented later on.

There are many retroviruses and examples include: murine leukaemia virus (MLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

There is also a family called lentiviruses including human immunodeficiency virus (HIV) and equine infectious anaemia virus (EIAV). Further details are given below.

A detailed list of retroviruses and lentiviruses may be found in Coffin et al (“Retroviruses” 1997 Cold Spring Harbour Laboratory Press Eds: J M Coffin, S M Hughes, H E Varmus pp 758-763).

Details on the genomic structure of some retroviruses may be found in the art. By way of example, details on HIV may be found from the NCBI Genbank (i.e. Genome Accession No. AF033819).

As indicated above, there has been considerable interest in the development of retroviral vector systems based on lentiviruses, a small subgroup of the retroviruses. This interest arises firstly from the notion of using HIV-based vectors to target anti-HIV therapeutic genes to HIV susceptible cells and secondly from the prediction that, because lentiviruses are able to infect non-dividing cells (Lewis & Emerman 1993 J. Virol. 68, 510), vector systems based on these viruses would be able to transduce non-dividing cells (e.g. Vile & Russel 1995 Brit. Med. Bull. 51, 12). Vector systems based on HIV have been produced (Buchschacher & Panganiban 1992 J. Virol. 66, 2731) and they have been used to transduce CD4+ cells and, as anticipated, non-dividing cells (Naldini et al, 1996 Science 272, 263). In addition lentiviral vectors enable very stable long-term expression of the gene of interest. This has been shown to be at least three months for transduced rat neuronal cells. The MLV based vectors were only able to express the gene of interest for six weeks.

HIV-based vectors produced to date result in an integrated provirus in the transduced cell that has HIV LTRs at its ends. This limits the use of these vectors as the LTRs have to be used as expression signals for any inserted gene unless an internal promoter is used. The use of internal promoters has significant disadvantages. The unpredictable outcome of placing additional promoters within the retroviral LTR transcription unit is well documented (Bowtell et al, 1988 J. Virol. 62, 2464; Correll et al, 1994 Blood 84, 1812; Emerman and Temin 1984 Cell 39, 459; Ghattas et al, 1991 Mol. Cell. Biol. 11, 5848; Hantzopoulos et al, 1989 PNAS 86, 3519; Hatzoglou et al, 1991 J. Biol. Chem 266, 8416; Hatzoglou et al, 1988 J. Biol. Chem 263, 17798; Li et al, 1992 Hum. Gen. Ther. 3, 381; McLachlin et al, 1993 Virol. 195, 1; Overell et al, 1988 Mol. Cell Biol. 8, 1803; Scharfman et al, 1991 PNAS 88, 4626; Vile et al, 1994 Gene Ther 1, 307; Xu et al, 1989 Virol. 171, 331; Yee et al, 1987 PNAS 84, 5197). The factors involved appear to include the relative position and orientation of the two promoters, the nature of the promoters and the expressed genes and any selection procedures that may be adopted. The presence of internal promoters can affect both the transduction titers attainable from a packaging cell line and the stability of the integrated vector.

HIV and other lentiviral LTRs have virus-specific requirements for gene expression. For example, the HIV LTR is not active in the absence of the viral Tat protein (Cullen 1995 AIDS 9, S19). It is desirable, therefore, to modify the LTRs in such a way as to change the requirements for gene expression. In particular tissue specific gene expression signals may be required for some gene therapy applications.

HIV vectors have a number of significant disadvantages which may limit their therapeutic application to certain diseases. HIV-1 has the disadvantage of being a human pathogen carrying potentially oncogenic proteins and sequences. There is the risk that introduction of vector particles produced in packaging cells which express HIV gag-pol will introduce these proteins into the patient leading to seroconversion. For these reasons, there is a need to develop lentiviral-based vectors which do not introduce HIV proteins into patients.

The use of MLV in the production of transgenic animals has also been proposed. However, the level of expression using this technique has been disappointing.

There is also a long felt need to generate disease models in which the transgene is regulatable and which can be generated efficiently.

Aspects of the present invention overcome these problems.

According to a first aspect of the present invention there is provided a method of producing a transgenic cell comprising introducing into a cell a non-primate lentiviral expression vector comprising a nucleotide of interest (NOI).

The present invention provides an efficient way of producing transgenic animals and which overcomes any potential difficulties associated with the use of primate lentiviruses.

Preferably, the non-primate lentiviral expression vector is derived from EIAV, FIV, BIV, CAEV or MVV, with EIAV being particularly preferred.

One of the advantages of the present invention is that the expression vector can be introduced in vivo or ex vivo. In one embodiment the method is carried out in vitro. In another embodiment, the cell is in utero.

Several methods for introducing foreign DNA into the germline of mammals have been developed. The techniques allow the mixing of cells from different embryos, i.e. chimera production, introducing pluripotent cells such as ES cells into developing embryos, micro-injecting DNA, and infection by retroviruses. Many of these techniques have the fundamental requirement of removing fertilised eggs or early embryos, culturing them in vitro and then returning them to foster mothers where further embryogenesis can proceed. In particular the production of transgenic animals by targeted insertion of DNA by homologous recombination in ES cells is a labour intensive and time-consuming process with, e.g. a turnaround time of 8 to 9 weeks from nuclear injection.

One major advantage of this embodiment of the present invention is the ability to avoid the need to remove, culture in vitro and then reimplant cells. It also avoids the intensive and time-consuming production of recombinant ES cells.

Indeed, a vast number of genes of unknown function are now available following large scale gene sequencing programmes. To develop therapeutic products from novel genomic targets, it will be necessary to correlate biology with gene sequence information. The present invention provides an efficient and effective in vivo method for assisting in the validation of targets.

We have also found that good levels of expression may be achieved using the present invention, and that the levels of expression, particularly with EIAV, are better than those achieved using MLV. This is surprising.

Another advantage of the present invention is its efficiency. Regulatable knock-out disease models can be efficiently produced though transduction with one vector, if desired, and few generations are required. Thus, the present invention meets a long felt want whose solution was not obvious at the time.

A further advantage of this aspect of the present invention is its flexibility; the lentiviral vector can be introduced throughout the development of the organism. Thus in one embodiment the cell is a perinatal cell, which could be an embryonic cell. In a particular aspect of this embodiment the embryonic cell is in utero. However, the method may be applied to any cell such as any somatic cell and also any cell which is capable of giving rise to a germ line change. Such cells include the germ cells, of course, but the present invention can also be applied to a cell which is involved either directly or indirectly in gametogenesis or fertilisation. We also include equivalent cells which are arrived at without direct fertilisation, e.g. through cell nuclear replacement techniques.

Preferably the cell is an oocyte, an oviduct cell, an ovarian cell, an ovum, an ES cell, a blastocyte, a spermatocyte, a spermatid, a spermatozoa, or a spermatogonia.

When seeking to achieve germ line changes, it will be appreciated that the earlier transduction occurs, the better as there is a greater chance of transducing germ cells.

A particular advantage with the use of lentiviral vectors is that it is possible to transduce non- or slowly-dividing cells, such as oocytes and sperm-forming cells.

Therefore according to another aspect of the present invention there is provided a method for producing a transgenic cell comprising introducing into a nondividing cell a lentiviral expression vector comprising an NOI. The lentiviral expression vector may be derived from a non-primate lentivirus, but may also be derived from a primate lentivirus such as HIV.

An important advantage of this aspect of the present invention is that cells do not have to be fertilised for transduction to be achieved.

By non-dividing cells we include cells which are capable of dividing but are non-dividing at a particular time.

The method is not limited to a particular cell type, but the cell is preferably a eukaryotic cell, such as an animal, preferably mammalian, or yeast cell. Examples of cells to which the present invention is applicable include murine, human, porcine, bovine, simian, ovine, equine, avian such as fowl, particularly chickens, insect or reptile or piscine cell. The cell may be from, e.g., C. elegans or drosophila.

In one embodiment, the cell is from a non-human organism.

Preferably the lentiviral expression vector is pseudotyped.

Preferably the lentiviral expression vector does not contain any functional accessory genes.

The NOI may be operably linked to a constitutive, tissue-specific or an inducible promoter.

Preferably, the NOI encodes and is capable of expressing a therapeutic protein, or encodes an antisense oligonucleotide or encodes a ribozyme.

In a preferred embodiment the NOI is capable of generating an RNA molecule capable of post-transcriptional silencing of a target gene. In this aspect of the present invention we provide a method of producing a transgenic cell comprising introducing into a cell a lentiviral expression vector comprising an NOI capable of generating an RNA molecule capable of post-transcriptional silencing of a target gene.

Preferably, the NOI is capable of generating a short RNA, a siRNA, a short hairpin RNA, a micro-RNA or a group I intron.

In one embodiment, expression of a short RNA, a siRNA, a short hairpin RNA, a micro-RNA is regulated by a tetracycline-responsive derivative of an RNA polymerase promoter.

In one embodiment the method comprising introducing at least one NOI which is capable of expressing a protein, preferably a therapeutic protein, and at least one NOI which is capable of generating an RNA molecule capable of post-transcriptional silencing of a target gene. The at least one NOI which is capable of expressing a protein, preferably a therapeutic protein, and the at least one NOI which is capable of generating an RNA molecule capable of post-transcriptional silencing of a target gene may be incorporated within the same or separate lentiviral expression vectors.

The lentiviral expression vector may be introduced into a target cell through administration via any convenient route of access, such as a cell of the umbilical cord, placenta, or amniotic fluid; or directly into an organ such as the uterus, gonad, brain, kidney, liver, heart, bone marrow, blood, central nervous system, or lung.

One problem associated with the production of transgenic animals for establishing disease models arises where the loss of expression in say a knock out mouse is lethal. In the methods of the present invention the NOI can be operably linked to a tissue-specific or an inducible promoter. This is particularly advantageous where ablation of gene expression is desired at a particular developmental stage or in a specific tissue.

The NOI may be expressed in the transgenic organism in a constitutive, tissue-specific or regulatable manner. Examples of cells where the NOI may be expressed include a cell of any organ or tissue, such as a cell of the brain, kidney, liver, heart, bone marrow, blood, central nervous system, or lung of said organism. The NOI may also be expressed at a particular developmental stage of the organism.

As discussed above, in a preferred embodiment of the present invention, said NOI encodes an RNA e.g., a short RNA, a siRNA, a short hairpin RNA or a micro-RNA capable of post-transcriptional silencing of a target gene.

The present invention also relates to transgenic animals derivable from such cells. As well as their use in disease models it will be appreciated that such transgenic animals can be used in the production of proteins, such a therapeutic proteins, e.g. insulin.

In a particular aspect of the present invention there is provided an egg derived from a transgenic avian. Thus according to this aspect of the present invention there is provided a method for generating a transgenic avian and/or egg comprising introducing into an avian cell a lentiviral expression vector comprising an NOI. In the case where the NOI encodes a protein, the protein can be cleanly and efficiently harvested from the transgenic egg expressing the protein.

However, we have recognised that there is a limit on the volume of protein which can be produced in an egg due to its size. We have now found that by silencing one or more of the genes encoding proteins which are naturally present in the egg it is possible to increase the yield of the protein expressed by the introduced NOI. These natural proteins may be down-regulated through the method of the present invention, i.e. through the use of a lentiviral expression vector encoding at least one NOI which is capable of generating an RNA molecule capable of post-transcriptional silencing of a natural egg gene. The at least one NOI which is capable of expressing a protein, preferably a therapeutic protein, and the at least one NOI which is capable of generating an RNA molecule capable of post-transcriptional silencing of the target egg gene may be incorporated within the same or separate lentiviral expression vectors. In a preferred embodiment of this aspect of the present invention the NOI capable of expressing the protein is placed under the control of a promoter which is native to the egg, such as the lysozyme promoter.

Thus the present invention provides a transgenic organism or egg comprising at least one NOI capable of expressing a protein, preferably a therapeutic protein, and at least one NOI which is capable of generating an RNA molecule capable of post-transcriptional silencing of a natural egg gene. As discussed above this second NOI may encode an RNA e.g., a short RNA, a siRNA, a short hairpin RNA or a micro-RNA capable of post-transcriptional silencing of a target gene.

Post-transcriptional gene silencing (PTGS) mediated by double-stranded dsRNA is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called small interfering or silencing RNAs (siRNAs) which are the sequence-specific mediators of mRNA degradation. In addition to siRNAs, the expression of short RNAs may act to redirect splicing (‘exon-skipping’) or polyadenylation or to inhibit translation.

Although viral vectors are efficient tools for in vivo gene delivery, the short length of RNA molecules involved in post-transcriptional gene silencing means that transcription of these RNAs using conventional expression cassettes is difficult. A further problem is that the use of viral vectors, e.g., lentiviral vectors, for generating transgenics to deliver the aforementioned RNA molecules which target a gene product with an important or essential function may result in death of the transgenic animal during development. An aspect of the present invention overcomes this problem by providing vectors in which transcription of polynucleotides e.g., siRNAs, are able to be regulated by use of an aptazyme. Indeed this aspect of the invention is not limited to methods involving lentiviral vectors and constitutes an independent aspect of the invention.

Thus, in a second aspect of the invention there is provided a vector comprising a first nucleotide sequence, wherein said first nucleotide sequence comprises:
(a) a second nucleotide sequence encoding an aptazyme; and
(b) a third nucleotide sequence capable of generating a polynucleotide;
   wherein (a) and (b) are operably linked and wherein the aptazyme is activatable to cleave a transcript of the first nucleotide sequence such that said polynucleotide is generated.

In other words there is provided a vector comprising a nucleic acid sequence, wherein said nucleic acid sequence comprises:
(c) a first nucleotide sequence encoding an aptazyme; and
(d) a second nucleotide sequence capable of generating a polynucleotide;
   wherein (a) and (b) are operably linked and wherein the aptazyme is activatable to cleave a transcript of the nucleic acid sequence such that said polynucleotide is generated.

Preferably the vector is a viral vector.

In a preferred embodiment of this aspect of the invention, the polynucleotide is an RNA molecule capable of modulating expression of a target gene. Preferably, the RNA molecule is selected from the group comprising siRNA, short hairpin RNA, microRNA, anti-sense RNA and a ribozyme.

Aptazymes are allosteric ribozymes. Aptamers are nucleic acid molecules which form structures which are able to bind a number of ligands including proteins and drug molecules. By replacing one helix of a ribozyme, e.g. a hammerhead ribozyme, with an aptamer it has been possible to create a catalytic RNA which is able to cleave a substrate (which may be itself) as the result of conformational change induced by the presence or absence of a ligand. Aptazymes which can be induced or inhibited by flavin mononucleotide (FMN) have been described (Soukup and Breaker 1999), as has an aptazyme which is inhibited by doxycycline (Piganaeu et al 2000).

In a further independent aspect of the present invention, cleavage induced by an aptazyme may be used to directly modulate expression of a NOI.

Thus, according to a third aspect of the present invention there is provided a vector comprising a first nucleotide sequence, wherein said first nucleotide sequence comprises:
(a) a second nucleotide sequence encoding an aptazyme; and
(b) a third nucleotide sequence comprising a NOI;
wherein (a) and (b) are operably linked and wherein the aptazyme is activatable to cleave the transcript of the first nucleotide sequence such that expression of said NOI is inhibited.

In other words the present invention provides a vector comprising a nucleic acid sequence, wherein said nucleic acid sequence comprises:
(c) a first nucleotide sequence encoding an aptazyme; and
(d) a second nucleotide sequence comprising a NOI;
wherein (a) and (b) are operably linked and wherein the aptazyme is activatable to cleave the transcript of the nucleic acid sequence such that expression of said NOI is inhibited.

Preferably the vector is a viral vector.

In one embodiment the aptazyme encoded by the above vector is activatable to cleave the transcript of the first nucleotide sequence at a position within the transcript of the third nucleotide sequence.

In a preferred embodiment the NOI encoded by the vector according to the third aspect of the present invention encodes a therapeutic protein.

In one embodiment of the second and third aspects of the present invention the aptazyme is activated by binding of a ligand to the aptazyme.

In another embodiment of the second and third aspects of the present invention the aptazyme is deactivated by binding of a ligand to the aptazyme.

In a preferred embodiment of the second and third aspects of the present invention the vector further comprises a fourth nucleotide sequence encoding a ligand capable of binding the aptazyme. The nucleotide sequence encoding the ligand may be operatively linked to a promoter.

The ligand may be selected from the group comprising polypeptides and fragments thereof, linear peptides, cyclic peptides, and nucleic acids which encode therefor, synthetic and natural compounds including low molecular weight organic or inorganic compounds and antibodies.

In preferred embodiments the ligand for use in these aspects of the invention is selected from the group comprising FMN, doxycycline and VEGF, tetracycline or glucose.

In another embodiment of the second and third aspects of the present invention (a) and (b), are operably linked to a promoter. Preferred promoters are selected from the group comprising RNA polymerase III (U6) promoters such as the U6 promoter as well as conventional RNA polymerase II promoters.

The promoter may be operably linked to at least one copy of a tetracycline responsive element (TRE), e.g., the Tet operator, such that transcription of the first nucleotide sequence is regulated by a tetracycline modulator and tetracycline or derivatives thereof.

In one embodiment, the vector according to the second and third aspects of the present invention comprises a fifth nucleotide sequence encoding a tetracycline modulator.

Although the production of the vector is preferably carried out under conditions which should minimise activity of the aptazyme, and hence unwanted destruction of the vector genome by self-cleavage, in a preferred embodiment the vector is configured as a split intron vector. This ensures that the full sequence of the aptazyme is only present in the transcript encoded by the provirus and not in the RNA genome present in the vector particle. An additional means of preventing formation of a potentially active aptazyme within the viral RNA genome is to use a promoter containing a sequence at its 3′ end which is able to base-pair with a part of the aptazyme such as to form a hairpin to prevent formation of active aptazyme within the viral RNA genome. Details of split intron vectors are described in WO 99/15683.

The vector according to the second and third aspects of the present invention may be derived from any suitable virus, for example a retrovirus, a lentivirus, an adenovirus, an adeno-associated vector, a herpes vector, a pox viral vector, a parvovirus vector or a baculoviral vector.

In a fourth aspect of the present invention there is provided a method of producing a transgenic cell using a vector of the second or third aspects of the present invention

According to a fifth aspect of the present invention there is provided a transgenic cell produced according to any of the methods of the present invention.

According to a sixth aspect of the present invention there is provided a transgenic organism which is generated from or obtainable by generation from a transgenic cell of the present invention.

According to a seventh aspect of the present invention there is provided a transgenic organism of the present invention wherein the NOI is expressed in a haematopoietic cell, (including monocytes, macrophages, lymphocytes, granulocytes, or progenitor cells of any of these); endothelial cell, tumour cell, stromal cell, astrocyte, or glial cell, muscle cell, epithelial cell, neuron, fibroblast, hepatocyte. astrocyte, kidney, liver, heart or lung cell.

In another aspect of the present invention there is provided a transgenic organism according to the present invention wherein the NOI is expressed in an oviduct cell, reproductive tract cell, albumin, haematopoietic cell, (including monocytes, macrophages, lymphocytes, granulocytes, or progenitor cells of any of these); endothelial cell, tumour cell, stromal cell, astrocyte, or glial cell, muscle cell, epithelial cell, neuron, fibroblast, hepatocyte. astrocyte, kidney, liver, heart or lung cell.

Various preferred features and embodiments of the present invention will now be described by way of non-limiting example and with reference to the accompanying drawings in which:

### DESCRIPTION OF THE FIGURES

FIG. 1 shows a liver and tissue histology after in utero injection of EIAV lentivirus and shows sections of mouse liver stained for the β-galactosidase marker gene 3, 7, 14, 28, 79 days and 6 months after foetal intravenous injection;

FIG. 2 shows tissue and histology after in utero injection of EIAV lentivirus and shows section of mouse liver, heart, skeletal muscle, lung, brain, and kidney stained for the β-galactosidase marker gene at variously 3, 7, 14 and 79 days after foetal intravenous injection;

FIG. 3 shows a mouse dorsal root ganglia stained for the β-galactosidase marker gene 7 days post foetal intraspinal injection of EIAV viral vector expressing nuclear localising LacZ;

FIG. 4 shows a section of a mouse dorsal root ganglia stained for the β-galactosidase marker gene days post foetal intraspinal injection of EIAV viral vector expressing nuclear localising LacZ;

FIG. 5 shows a section of mouse liver stained for the β-galactosidase marker gene 7 days post foetal intravenous injection of EIAV viral vector expression nuclear localising LacZ;

FIG. 6 shows a mouse renal glomeruli and a section thereof stained for the β-galactosidase marker gene 7 days post foetal intravenous injection of EIAV viral vector expression nuclear localising LacZ;

FIG. 7 shows a mouse pancreas and a section thereof stained for the β-galactosidase marker gene 7 days post foetal intravenous injection of EIAV viral vector expression nuclear localising LacZ;

FIG. 8 shows mouse skeletal muscle stained for the β-galactosidase marker gene 7 days post foetal intramuscular injection of EIAV viral vector expression nuclear localising LacZ;

FIG. 9 shows a mouse diaphragm and planar and transverse sections thereof stained for the β-galactosidase marker gene two weeks post foetal intraperitoneal injection of EIAV viral vector expression LacZ;

FIG. 10 shows a mouse leg and planar and transverse sections thereof stained for the β-galactosidase marker gene two weeks post foetal intramuscular injection of EIAV viral vector expression nuclear localising LacZ;

FIG. 11 shows X-Gal visualisation for β-galactosidase 96 hourse after intra-thoracic and intra-peritoneal injection of EIAV viral vector. FIG. 11A shows a sagittal section with the viscera removed. The diaphragm has been excised and is viewed anteriorly in FIG. 11B;

FIG. 12 shows schematic representations of EIAV genomes with sizes. These may be used for transfection in the present invention. Upon transfection the 3′ LTR will be copied to the 5′ LTR;

FIG. 13 shows the nucleic acid sequence of pONY8.1G;

FIG. 14 shows the nucleic acid sequence of pONY8.4ZCG;

FIG. 15 shows the nucleic acid sequence of pONY8.4GCZ;

FIG. 16 is a schematic representation of the hybrid U3 region of a vector for use in the present invention;

FIG. 17 shows the nucleic acid sequence of this hybrid LTR;

FIG. 18 shows the nucleic acid sequence of pONY8.1ZHyb;

FIG. 19 is a schematic representation of pONY8.1ZHyb;

FIGS. 20 (a)-(c) show expression cassettes for use in RNAi applications;

FIG. 21 (a) shows an expression cassette for use in mediating aptazyme regulated siRNA gene silencing;

FIG. 21 (b) shows the structure of the transcripts of the expression cassette of FIG. 24a.

FIG. 22 shows an expression cassette for use in hypoxically inducing silencing of VEGF by siRNAs;

FIG. 23 (a) shows an expression cassette comprising an RNA polymerase II promoter for expressing aptazyme regulated short hairpin;

FIG. 23 (b) shows an expression cassette comprising an RNA polymerase II promoter for expressing aptazyme regulated antisense siRNA;

FIG. 24 (a) shows an expression cassette for use in mediating aptazyme regulated insulin expression;

FIG. 24 (b) shows an expression cassette for use in mediating aptazyme regulated Factor IX expression;

FIG. 25 (a) shows a schematic of a split intron strategy to avoid self-cleavage of RNA genome;

FIG. 25 (b) shows an expression cassette for use in a split intron strategy;

FIG. 26 shows a schematic of a double hairpin strategy to avoid self-cleavage of RNA genome.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc.

One aspect of the present invention relates to a method of producing a transgenic cell using a non-primate lentiviral expression vector and a transgenic organism which is obtainable from the transgenic cell or of which the transgenic cell forms part. More particularly, this aspect of the present invention relates to a lentiviral vector useful in gene therapy and in the production of disease models. The development of disease models, e.g. transgenic “knockout” mice, has greatly benefited studies of gene function, with particular relevance in establishing mammalian models of genetic disease.

Gene therapy includes any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology (Ed Robert Meyers, Pub VCH, such as pages 556-558).

By way of further example, gene therapy also provides a means by which any one or more of: a nucleotide sequence, such as a gene, can be applied to replace or supplement a defective gene; a pathogenic gene or gene product can be eliminated; a new gene can be added in order, for example, to create a more favourable phenotype; cells can be manipulated at the molecular level to treat cancer (Schmidt-Wolf and Schmidt-Wolf, 1994, Annals of Hematology 69:273-279) or other conditions—such as immune, cardiovascular, neurological, inflammatory or infectious disorders; antigens can be manipulated and/or introduced to elicit an immune response—such as genetic vaccination.

A transgenic organism is an organism which includes in at least one of its cells a nucleotide of interest (NOI). In one embodiment the cell is a germline cell. In another embodiment, the cell is a somatic cell. More particularly, the NOI has been introduced experimentally, e.g. using cDNA technology.

The NOI is commonly referred to as a “transgene”, i.e. a gene that is inserted into the cell in such a way that ensures its function. When the gene is inserted into a germ line gene it should function, replicate and be transmitted as a normal gene.

The present invention encompasses chimeras and mosaics.

A “chimera” is an organism composed of a mixture of genetically different cells.

A “mosaic” is an organism in which the transgene is incorporated into the genome after the first cell division. The organism will be mosaic as different cells will have different sites of integration.

A transgenic organism of the invention is preferably a multicellular eukaryotic organism, such as an animal or a plant, or a fungus, or a unicellular eukaryotic organism such as a yeast.

Then organism is preferably an animal, more preferably a mammal.

The first aspect of the present invention employs a non-primate lentiviral expression vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host cell for the purpose of replicating the vectors comprising a segment of DNA. Examples of vectors used in recombinant DNA techniques include but are not limited to plasmids, chromosomes, artificial chromosomes or viruses.

The term “expression vector” means a construct capable of in vivo or in vitro/ex vivo expression.

The lentiviral vector used in aspects of the present invention is capable of transducing a target non-dividing cell. One advantage of this feature is that since freshly isolated oocytes are quiescent, transduction rates may be enhanced by the use of lentiviral rather than retroviral vectors.

In a typical vector for use in the method of the present invention, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target non-dividing host cell and/or integrating its genome into a host genome.

Preferrably the viral vector capable of transducing a target non-dividing or slowly dividing cell is a lentiviral vector.

Lentivirus vectors are part of a larger group of retroviral vectors. A detailed list of lentiviruses may be found in Coffin et al (“Retroviruses” 1997 Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al 1992 EMBO. J 11: 3053-3058; Lewis and Emerman 1994 J. Virol. 68: 510-516). In contrast, other retroviruses—such as MLV—are unable to infect non-dividing or slowly dividing cells such as those that make up, for example, muscle, brain, lung and liver tissue.

A “non-primate” vector, as used herein in some aspects of the present invention, refers to a vector derived from a virus which does not primarily infect primates, especially humans. Thus, non-primate virus vectors include vectors which infect non-primate mammals, such as dogs, sheep and horses, reptiles, birds and insects.

A lentiviral or lentivirus vector, as used herein, is a vector which comprises at least one component part derivable from a lentivirus. Preferably, that component part is involved in the biological mechanisms by which the vector infects cells, expresses genes or is replicated. The term “derivable” is used in its normal sense as meaning the sequence need not necessarily be obtained from a retrovirus but instead could be derived therefrom. By way of example, the sequence may be prepared synthetically or by use of recombinant DNA techniques.

The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). Preferably the lentivirus is an EIAV. Equine infectious anaemia virus infects all equidae resulting in plasma viremia and thrombocytopenia (Clabough, et al. 1991. J Virol. 65:6242-51). Virus replication is thought to be controlled by the process of maturation of monocytes into macrophages.

In one embodiment the viral vector is derived from EIAV. EIAV has the simplest genomic structure of the lentiviruses and is particularly preferred for use in the present invention. In addition to the gag, pol and env genes EIAV encodes three other genes: tat, rev, and S2. Tat acts as a transcriptional activator of the viral LTR (Derse and Newbold 1993 Virology. 194:530-6; Maury, et al 1994 Virology. 200:632-42) and Rev regulates and coordinates the expression of viral genes through rev-response elements (RRE) (Martarano et al 1994 J Virol. 68:3102-11). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of tat spliced to the env coding sequence at the start of the transmembrane protein.

In addition to protease, reverse transcriptase and integrase non-primate lentiviruses contain a fourth pol gene product which codes for a dUTPase. This may play a role in the ability of these lentiviruses to infect certain non-dividing cell types.

The viral RNA of this aspect of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

Thus the present invention employs a DNA transcription unit comprising a promoter and optionally an enhancer capable of directing expression of a non-primate lentiviral vector genome.

Transcription units as described herein comprise regions of nucleic acid containing sequences capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

Preferred vectors for use in accordance with one aspect of the present invention are recombinant non-primate lentiviral vectors.

The term “recombinant lentiviral vector” (RLV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication. The vector of the present invention may be configured as a split-intron vector. A split intron vector is described in PCT patent application WO 99/15683.

Preferably the lentiviral vector of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO98/17815.

A minimal lentiviral genome for use in the present invention will therefore comprise (5′) R-U5—one or more first nucleotide sequences—U3-R (3′). However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included. However the requirement for rev and RRE may be reduced or eliminated by codon optimisation. Further details of this strategy can be found in our WO01/79518.

In one embodiment of the present invention, the lentiviral vector is a self-inactivating vector.

By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

In our WO99/32646 we give details of features which may advantageously be applied to the present invention. In particular, it will be appreciated that the non-primate lentivirus genome (1) preferably comprises a deleted gag gene wherein the deletion in gag removes one or more nucleotides downstream of about nucleotide 350 or 354 of the gag coding sequence; (2) preferably has one or more accessory genes absent from the non-primate lentivirus genome; (3) preferably lacks the tat gene but includes the leader sequence between the end of the 5′ LTR and the ATG of gag; and (4) combinations of (1), (2) and (3). In a particularly preferred embodiment the lentiviral vector comprises all of features (1) and (2) and (3).

The non-primate lentiviral vector may be a targeted vector. The term “targeted vector” refers to a vector whose ability to infect/transfect/transduce a cell or to be expressed in a host and/or target cell is restricted to certain cell types within the host organism, usually cells having a common or similar phenotype.

Target cells for gene therapy using retroviral vectors include but are not limited to haematopoietic cells, (including monocytes, macrophages, lymphocytes, granulocytes, or progenitor cells of any of these); endothelial cells, tumour cells, stromal cells, astrocytes, or glial cells, muscle cells, epithelial cells, neurons, fibroblasts, hepatocyte. astrocyte, kidney, liver, heart and lung cells.

The vector may be pseudotyped with any molecule of choice, including but not limited to envelope glycoproteins (wild type or engineered variants or chimeras) of VSV-G, rabies, Mokola, MuLV, LCMV, Sendai, Ebola.

Although the first aspect of the present invention is directed to a method which, in particular, uses lentiviral vectors, other aspects of the present invention may employ other viral expression vectors. Viral vectors according to these aspects include but are not limited to a retroviral vector, a lentiviral vector, an adenoviral vector, an adeno-associated viral vector, a herpes viral vector, a pox viral vector, a parvoviral vector or a baculoviral vector.

The retroviral vector employed in the aspects of the present invention may be derived from or may be derivable from any suitable retrovirus. A large number of different retroviruses have been identified. Examples include: murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

The adenovirus is a double-stranded, linear DNA virus that does not go through an RNA intermediate. There are over 50 different human serotypes of adenovirus divided into 6 subgroups based on the genetic sequence homology. The natural target of adenovirus is the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Viral gene expression can be divided into early (E) and late (L) phases. The late phase is defined by the onset of viral DNA replication. Adenovirus structural proteins are generally synthesised during the late phase. Following adenovirus infection, host cellular mRNA and protein synthesis is inhibited in cells infected with most serotypes. The adenovirus lytic cycle with adenovirus 2 and adenovirus 5 is very efficient and results in approximately 10,000 virions per infected cell along with the synthesis of excess viral protein and DNA that is not incorporated into the virion. Early adenovirus transcription is a complicated sequence of interrelated biochemical events but it entails essentially the synthesis of viral RNAs prior to the onset of DNA replication.

The organisation of the adenovirus genome is similar in all of the adenovirus groups and specific functions are generally positioned at identical locations for each serotype studied. Early cytoplasmic messenger RNAs are complementary to four defined, noncontiguous regions on the viral DNA. These regions are designated E1-E4. The early transcripts have been classified into an array of intermediate early (E1a), delayed early (E1b, E2a, E2b, E3 and E4), and intermediate regions.

The early genes are expressed about 6-8 hours after infection and are driven from 7 promoters in gene blocks E1-4.

Adenoviruses may be converted for use as vectors for gene transfer by deleting the E1 gene, which is important for the induction of the E2, E3 and E4 promoters. The E1-replication defective virus may be propagated in a cell line that provides the E1 polypeptides in trans, such as the human embryonic kidney cell line 293. A therapeutic gene or genes can be inserted by recombination in place of the E1 gene. Expression of the gene is driven from either the E1 promoter or a heterologous promoter.

Even more attenuated adenoviral vectors have been developed by deleting some or all of the E4 open reading frames (ORFs). However, certain second generation vectors appear not to give longer-term gene expression, even though the DNA seems to be maintained. Thus, it appears that the function of one or more of the E4 ORFs may be to enhance gene expression from at least certain viral promoters carried by the virus.

An alternative approach to making a more defective virus has been to “gut” the virus completely maintaining only the terminal repeats required for viral replication. The “gutted” or “gutless” viruses can be grown to high titres with a first generation helper virus in the 293 cell line but it has been difficult to separate the “gutted” vector from the helper virus.

The adenovirus provides advantages as a vector for identifying candidate modulating moieties over other gene therapy vector systems for the following reasons:

It is a double stranded DNA nonenveloped virus that is capable of in vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons.

Adenoviral vectors are also capable of transducing non dividing cells. This is very important for diseases, such as cystic fibrosis, in which the affected cells in the lung epithelium, have a slow turnover rate. In fact, several trials are underway utilising adenovirus-mediated transfer of cystic fibrosis transporter (CFTR) into the lungs of afflicted adult cystic fibrosis patients.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, it functions episomally (independently from the host genome) as a linear genome in the host nucleus. Hence the use of recombinant adenovirus alleviates the problems associated with random integration into the host genome.

Pox viral vectors may be used in accordance with aspects of the present invention, as large fragments of DNA are easily cloned into its genome and recombinant attenuated vaccinia variants have been described (Meyer, et al., 1991, J. Gen. Virol. 72: 1031-1038, Smith and Moss 1983 Gene, 25:21-28).

Examples of pox viral vectors include but are not limited to leporipoxvirus: Upton, et al J. Virology 60:920 (1986) (shope fibroma virus); capripoxvirus: Gershon, et al J. Gen. Virol. 70:525 (1989) (Kenya sheep-1); orthopoxvirus: Weir, et al J. Virol 46:530 (1983) (vaccinia); Esposito, et al Virology 135:561 (1984) (monkeypox and variola virus); Hruby, et al PNAS, 80:3411 (1983) (vaccinia); Kilpatrick, et al Virology 143:399 (1985) (Yaba monkey tumour virus); avipoxvirus: Binns, et al J. Gen. Virol 69:1275 (1988) (fowlpox); Boyle, et al Virology 156:355 (1987) (fowlpox); Schnitzlein, et al J. Virological Method, 20:341 (1988) (fowlpox, quailpox); entomopox (Lytvyn, et al J. Gen. Virol 73:3235-3240 (1992)].

Poxvirus vectors are used extensively as expression vehicles for genes of interest in eukaryotic cells. Their ease of cloning and propagation in a variety of host cells has led, in particular, to the widespread use of poxvirus vectors for expression of foreign protein and as delivery vehicles for vaccine antigens (Moss, B. 1991, Science 252: 1662-7).

Pox viruses which may be used in accordance with aspects of the present invention include but are not limited to recombinant pox viral vectors such as fowl pox virus (FPV), entomopox virus, vaccinia virus such as NYVAC, canarypox virus, MVA or other non-replicating viral vector systems such as those described for example in WO 95/30018. Pox virus vectors have also been described where at least one immune evasion gene has been deleted (see WO 00/29428).

As indicated above, a nucleotide sequence used in a method of the present invention is inserted into a vector which is operably linked to a control sequence that is capable of providing for the expression of the coding sequence by the host cell, i.e. the vector is an expression vector. The NOI produced by a host recombinant cell may be secreted or may be contained intracellularly depending on the sequence and/or the vector used.

The heterologous gene, i.e. NOI, may be any allelic variant of a wild-type gene, or it may be a mutant gene. The term “gene” is intended to cover nucleic acid sequences which are capable of being at least transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns). However, it is generally preferred to use cDNA because it is expressed more efficiently since intron removal is not required.

Suitable NOI coding sequences include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives thereof (such as with an associated reporter group). When included, such coding sequences may be typically operatively linked to a suitable promoter, which may be a promoter driving expression of a ribozyme(s), or a different promoter or promoters.

Suitable NOIs for use in the present invention in the treatment or prophylaxis of cancer include NOIs encoding proteins which: destroy the target cell (for example a ribosomal toxin), act as: tumour suppressors (such as wild-type p53); activators of anti-tumour immune mechanisms (such as cytokines, co-stimulatory molecules and immunoglobulins); inhibitors of angiogenesis; or which provide enhanced drug sensitivity (such as pro-drug activation enzymes); indirectly stimulate destruction of target cell by natural effector cells (for example, strong antigen to stimulate the immune system or convert a precursor substance to a toxic substance which destroys the target cell (for example a prodrug activating enzyme)). Encoded proteins could also destroy bystander tumour cells (for example with secreted antitumour antibody-ribosomal toxin fusion protein), indirectly stimulate destruction of bystander tumour cells (for example cytokines to stimulate the immune system or procoagulant proteins causing local vascular occlusion) or convert a precursor substance to a toxic substance which destroys bystander tumour cells (e.g. an enzyme which activates a prodrug to a diffusible drug).

NOI(s) may be used which encode antisense transcripts or ribozymes which interfere with expression of cellular genes for tumour persistence (for example against aberrant myc transcripts in Burkitts lymphoma or against bcr-abl transcripts in chronic myeloid leukemia). The use of combinations of such NOIs is also envisaged.

For further information on the nature of therapeutic genes see WO95/21927 and WO98/15294.

Suitable NOIs for use in the treatment or prevention of ischaemic heart disease include NOIs encoding plasminogen activators. Suitable NOIs for the treatment or prevention of rheumatoid arthritis or cerebral malaria include genes encoding anti-inflammatory proteins, antibodies directed against tumour necrosis factor (TNF) alpha, and anti-adhesion molecules (such as antibody molecules or receptors specific for adhesion molecules).

Examples of hypoxia regulatable therapeutic NOIs can be found in WO95/21927.

The NOI coding sequence may encode a fusion protein or a segment of a coding sequence.

Instead of, or as well as, being selectively expressed in target tissues, the NOI or NOIs may encode a pro-drug activating enzyme or enzymes which have no significant effect or no deleterious effect until the individual is treated with one or more pro-drugs upon which the enzyme or enzymes act. In the presence of the active NOI, treatment of an individual with the appropriate pro-drug leads to enhanced reduction in tumour growth or survival.

A pro-drug activating enzyme may be delivered to a tumour site for the treatment of a cancer. In each case, a suitable pro-drug is used in the treatment of the patient in combination with the appropriate pro-drug activating enzyme. An appropriate pro-drug is administered in conjunction with the vector. Examples of pro-drugs include: etoposide phosphate (with alkaline phosphatase); 5-fluorocytosine (with cytosine deaminase); doxorubicin-N-p-hydroxyphenoxyacetamide (with penicillin-V-amidase); para-N-bis(2-chloroethyl) aminobenzoyl glutamate (with carboxypeptidase G2); cephalosporin nitrogen mustard carbamates (with β-lactamase); SR4233 (with P450 Reductase); ganciclovir (with HSV thymidine kinase); mustard pro-drugs with nitroreductase and cyclophosphamide (with P450).

Examples of suitable pro-drug activating enzymes for use in the invention include a thymidine phosphorylase which activates the 5-fluoro-uracil pro-drugs capcetabine and furtulon; thymidine kinase from herpes simplex virus which activates ganciclovir; a cytochrome P450 which activates a pro-drug such as cyclophosphamide to a DNA damaging agent; and cytosine deaminase which activates 5-fluorocytosine. Preferably, an enzyme of human origin is used.

Suitable NOIs for use in the treatment or prevention of ischaemic heart disease include NOIs encoding plasminogen activators. Suitable NOIs for the treatment or prevention of rheumatoid arthritis or cerebral malaria include genes encoding anti-inflammatory proteins, antibodies directed against tumour necrosis factor (TNF) alpha, and anti-adhesion molecules (such as antibody molecules or receptors specific for adhesion molecules).

The expression products encoded by the NOIs may be proteins which are secreted from the cell. Alternatively the NOI expression products are not secreted and are active within the cell. In either event, it is preferred for the NOI expression product to demonstrate a bystander effect or a distant bystander effect; that is the production of the expression product in one cell leading to the killing of additional, related cells, either neighbouring or distant (e.g. metastatic), which possess a common phenotype.

Where macrophages or other haematopoietic cells are used, NOIs may be used which encode, for example, cytokines. These would serve to direct the subsequent differentiation of the haematopoietic stemp cells (HSCs) containing a therapeutic NOI. Suitable cytokines and growth factors include but are not limited to: ApoE, Apo-SAA, BDNF, Cardiotrophin-1, EGF, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1ac, IL-1α, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ and HCCl.

For some applications, a combination of some of these cytokines may be preferred, in particular a combination which includes IL-3, IL-6 and SCF, for the maintenance and expansion of stem cell populations. For differentiation in vitro, further cytokines may be added such as GM-CSF and M-CSF to induce differentiation of macrophages or GM-CSF and G-CSF to obtain neutrophils. On reintroduction of the engineered cells into the individual from whom they were derived, the body's own mechanisms then permit the cells or their differentiated progeny to migrate into the affected area e.g. the tumour.

Optionally, another NOI may be a suicide gene, expression of which in the presence of an exogenous substance results in the destruction of the transfected or transduced cell. An example of a suicide gene includes the herpes simplex virus thymidine kinase gene (HSV tk) which can kill infected and bystander cells following treatment with ganciclovir.

Optionally another NOI may be a targeting protein (such as an antibody to the stem cell factor receptor (WO-A-92/17505; WO-A-92/21766)). For example, recombinant (ecotropic) retroviruses displaying an antibody (or growth factor or peptide) against a receptor present on HSCs (CD34 or stem cell factor, for example) might be used for targeted cell delivery to these cells, either ex vivo by incubating unfractionated bone marrow with virus or by intravenous delivery of virus.

NOIs may also include marker genes (for example encoding β-galactosidase or green fluorescent protein) or genes whose products regulate the expression of other genes. In addition, NOIs may comprise sequences coding fusion protein partners in frame with a sequence encoding a protein of interest. Examples of fusion protein partners include the DNA binding or transcriptional activation domain of GAL4, a 6×His tag and β-galactosidase. It may also be desirable to add targeting sequences to target proteins encoding by NOIs to particular cell compartments or to secretory pathways. Such targeting sequences have been extensively characterised in the art.

In one embodiment, at least one NOI, operably linked to a bacterial HRE according to the present invention encodes an oxygen-responsive bacterial transcriptional regulatory protein such as FNR. Such a construct will provide an autoregulated system since in the presence of hypoxia, expression of the bacterial transcriptional regulatory protein from the HRE construct will increase and serve to further increase transcription from the HRE construct and other HRE constructs present.

In one preferred embodiment, the NOI encodes a ribozyme. Ribozymes are RNA molecules that can function to catalyse specific chemical reactions within cells without the obligatory participation of proteins. For example, group I ribozymes take the form of introns which can mediate their own excision from self-splicing precursor RNA. Other ribozymes are derived from self-cleaving RNA structures which are essential for the replication of viral RNA molecules. Like protein enzymes, ribozymes can fold into secondary and tertiary structures that provide specific binding sites for substrates as well as cofactors, such as metal ions. Examples of such structures include hammerhead, hairpin or stem-loop, pseudoknot and hepatitis delta antigenomic ribozymes have been described.

Each individual ribozyme has a motif which recognises and binds to a recognition site in a target RNA. This motif takes the form of one or more “binding arms” but generally two binding arms. The binding arms in hammerhead ribozymes are the flanking sequences Helix I and Helix III which flank Helix II. These can be of variable length, usually between 6 to 10 nucleotides each, but can be shorter or longer.

The length of the flanking sequences can affect the rate of cleavage. For example, it has been found that reducing the total number of nucleotides in the flanking sequences from 20 to 12 can increase the turnover rate of the ribozyme cleaving a HIV sequence, by 10-fold (Goodchild, J V K, 1991 Arch Biochem Biophys 284: 386-391). A catalytic motif in the ribozyme Helix II in hammerhead ribozymes cleaves the target RNA at a site which is referred to as the cleavage site. Whether or not a ribozyme will cleave any given RNA is determined by the presence or absence of a recognition site for the ribozyme containing an appropriate cleavage site.

Each type of ribozyme recognizes its own cleavage site. The hammerhead ribozyme cleavage site has the nucleotide base triplet GUX directly upstream where G is guanine, U is uracil and X is any nucleotide base. Hairpin ribozymes have a cleavage site of BCUGNYR, where B is any nucleotide base other than adenine, N is any nucleotide, Y is cytosine or thymine and R is guanine or adenine. Cleavage by hairpin ribozymes takes places between the G and the N in the cleavage site.

More details on ribozymes may be found in “Molecular Biology and Biotechnology” (Ed. R A Meyers 1995 VCH Publishers Inc p 831-8320 and in “Retroviruses” (Ed. J M Coffin et al 1997 Cold Spring Harbour Laboratory Press pp 683).

Expression of the ribozyme may be induced in all cells, but will only exert an effect in those in which the target gene transcript is present.

Alternatively, instead of preventing the association of the components directly, the substance may suppress the biologically available amount of a polypeptide of the invention. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

In another preferred embodiment, the NOI comprises an siRNA. Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called small interfering or silencing RNAs (siRNAs) which are the sequence-specific mediators of mRNA degradation. In differentiated mammalian cells dsRNA >30 bp has been found to activate the interferon response leading to shut-down of protein synthesis and non-specific mRNA degradation (Stark et al 1998). However this response can be bypassed by using 21 nt siRNA duplexes (Elbashir et al 2001, Hutvagner et al 2001) allowing gene function to be analysed in cultured mammalian cells.

In one embodiment an RNA polymerase III promoter, e.g., U6, whose activity is regulated by the presence of tetracycline may be used to regulate expression of the siRNA (Ohkawa et al, 2000).

In another embodiment the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme (Lagos-Quintana et al, 2001).

In a further embodiment the NOI comprises double-stranded interfering RNA in the form of a hairpin. The short hairpin may be expressed from a single promoter, e.g., U6. In an alternative embodiment an effective RNAi may be mediated by incorporating two promoters, e.g., U6 promoters, one expressing a region of sense and the other the reverse complement of the same sequence of the target. This is described in Example 9. In a further embodiment effective or double-stranded interfering RNA may be mediated by using two opposing promoters to transcribe the sense and antisense regions of the target from the forward and complementary strands of the expression cassette. These embodiments are described further in Example 9

In another embodiment the NOI may encode a short RNA which may act to redirect splicing (‘exon-skipping’) or polyadenylation or to inhibit translation. In the case of muscular dystrophy frame-shifting mutations in the dystrophin gene lead to a more severe Duchenne muscular dystrophy (DMD) phenotype than those which do not disrupt the translational reading frame (Becker muscular dystrophy). Antisense sequences targeted to induce skipping of exon 46 have been found to be effective in restoring dystrophin expression from the endogenous gene in DMD patient-derived muscle cells (van Deutekom et al 2001). Re-direction of polyadenylation by targeting antisense oligonucleotides to the 3′ most polyadenylation site of E-selectin has been shown to re-direct polyadenylation to cryptic upstream sites resulting in transcripts with fewer instability sequences thereby increasing mRNA stability and altering protein expression (Vickers et al 2001). In this way the use of antisense can be applied to increase the abundance of a message. Targeting to sites crucial for initiation of translation is also possible, in this case the mRNA abundance is increased but protein levels decrease.

The NOI may be under the expression control of an expression regulatory element, usually a promoter or a promoter and enhancer. The enhancer and/or promoter may be preferentially active in a hypoxic or ischaemic or low glucose environment, such that the NOI is preferentially expressed in the particular tissues of interest, such as in the environment of a tumour, arthritic joint or other sites of ischaemia. Thus any significant biological effect or deleterious effect of the NOI on the individual being treated may be reduced or eliminated. The enhancer element or other elements conferring regulated expression may be present in multiple copies. Likewise, or in addition, the enhancer and/or promoter may be preferentially active in one or more specific cell types—such as any one or more of macrophages, endothelial cells or combinations thereof. Further examples include respiratory airway epithelial cells, hepatocytes, muscle cells, cardiac myocytes, synoviocytes, primary mammary epithelial cells and post-mitotically terminally differentiated non-replicating cells such as macrophages and neurons.

The term “operably linked” means that the components described are in a relationship permitting them to function in their intended manner. A library comprising a regulatory sequence “operably linked” to a reporter sequence is ligated in such a way that expression of the nucleic acid reporter sequence is achieved under conditions compatible with the control sequences.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

The promoter and enhancer of the transcription units encoding the secondary delivery system are preferably strongly active, or capable of being strongly induced, in the primary target cells under conditions for production of the secondary delivery system. The promoter and/or enhancer may be constitutively efficient, or may be tissue or temporally restricted in their activity. Examples of temporally restricted promoters/enhancers are those which are responsive to ischaemia and/or hypoxia, such as hypoxia response elements or the promoter/enhancer of a grp78 or a grp94 gene. One preferred promoter-enhancer combination is a human cytomegalovirus (hCMV) major immediate early (MIE) promoter/enhancer combination.

In one preferred embodiment the combined use of a strong constitutive promoter such as CMV, or house-keeping promoter such as PGK, and the Tet-regulation system may be used for control of gene expression. In addition to the Tet system other inducible systems include the metallothionein, hsp68, lacZ, and SV40 T antigen systems.

Transactivating factors may be employed through use of two transgenic lines, namely one line which expresses the NOI under promoter “a”, and a second line which expresses the transactivating factor “b” of promoter “a”.

In another embodiment use may be made of the FLP recombinase system in which an inactive transgene is converted into the active form in a recombination event mediated by yeast FLP recombinase. Use may also be made of the bacteriophage P1 Cre recombinase system, which allows genes to be silenced in particular cell or tissue types and at specific times of the organisms development.

Ubiquitous expression may be achieved using promoters from housekeeping genes, such as beta-actin, mouse metallothionein, HMGCR and histone H4.

Preferably the promoters of the present invention are tissue specific. That is, they are capable of driving transcription of an NOI in one tissue while remaining largely “silent” in other tissue types.

The term “tissue specific” means a promoter which is not restricted in activity to a single tissue type but which nevertheless shows selectivity in that they may be active in one group of tissues and less active or silent in another group.

The level of expression of an NOI under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers.

Promoters suitable for cardiac-specific expression include the promoter from the murine cardiac α-myosin heavy chain (MHC) gene. Suitable vascular endothelium-specific promoters include the Et-1 promoter and von Willebrand factor promoter.

Prostate specific promoters include the 5′flanking region of the human glandular kallikrein-1 (hKLK2) gene and the prostate specific antigen (hKLK3).

Examples of promoters/enhancers which are cell specific include a macrophage-specific promoter or enhancer, such as CSF-1 promoter-enhancer, or elements from a mannose receptor gene promoter-enhancer (Rouleux et al 1994 Exp Cell Res 214:113-119). Alternatively, promoter or enhancer elements which are preferentially active in neutrophils, or a lymphocyte-specific enhancer such as an IL-2 gene enhancer, may be used.

Moreover, the NOI may be placed under the control of one or more sequences which confer developmentally-regulated expression. This will result in the NOIs being activated at a given stage in the development of the transgenic organism or its progeny.

Transcription of a NOI may be regulated by use of aptazymes. An aptazyme operably linked to a NOI may be activatable to cleave the transcript such that the NOI may be expressed following release of the NOI from the transcript. In a preferred embodiment the NOI is selected form the group comprising siRNA, short hairpin RNA, microRNA and anti-sense RNA. For example, the addition of an aptazyme 5′ of siRNA encoded by a short hairpin may allow the regulated induction (or inhibition) of self-cleavage of the transcript separating the hairpin from the aptazyme structure and hence activating silencing. Ligands specific for the aptamer may be supplied exogenously, expressed endogenously or from the same vector. For example a protein ligand whose expression is controlled by the hypoxic response element (HRE) would only be synthesised under hypoxic conditions. If this were to activate cleavage of an aptazyme which released a short hairpin targeted to vascular endothelial growth factor (VEGF), VEGF would be specifically down-regulated in hypoxia which would be therapeutically beneficial in a number of diseases including proliferative diabetic retinopathy. Alternatively the ligand for the aptamer could be VEGF itself.

In a further aspect of the invention, cleavage induced by the aptazyme may directly modulate expression of a NOI. The use of aptazymes in this way encompasses post-transcriptional regulation of a NOI according to the invention. The aptazyme may be activated (or inhibited) by the addition/removal of the appropriate ligand inducing cleavage of the transcript such that NOI expression is inhibited. The aptazyme may cleave the transcript at for example the codon for the initiator methionine, or a UTR resulting in a transcript lacking either cap and/or poly-adenosine tail which will be subsequently degraded prior to translation.

This provides a means of shutting off synthesis of a NOI, for example a therapeutic gene such as Factor IX, if levels are too high. Expression of the transgene may be engineered to be self-regulating in this way. For example an aptazyme whose activity is modulated by glucose binding could be designed such that high level expression of insulin occurs only when blood glucose levels are high. If glucose levels fall below a threshold level then the aptazyme is actived and the insulin transgene transcript destroyed. An aptazyme which is regulated by doxycycline may be used to regulate the expression of NOIs both in vitro and in vivo by the administration of doxycycline.

The Tet-regulation system may be used to control expression of the aptazyme to provide an additional level of control. Tet operator sequences inserted downstream of the promoter may repress transcription in the presence of the Tet repressor protein when doxycycline is removed, thereby preventing de novo transcription. As the aptazyme is active in the absence of doxycycline any existing transcripts will be cleaved and degraded.

The development of transgenic ‘knockout’ mouse technology has greatly benefited studies of gene function, with particular relevance in establishing mammalian models of genetic disease. Current technology is, however, limiting in certain cases. For example many genes, often those of medical significance, are essential for viability. In such cases pups die during embryonic development or soon after birth. The present invention provides an effective transgenic method for regulatable gene ablation such that the production of a protein of interest may be switched off at the desired developmental stage, facilitating the generation of disease models in adult mammals. The transgenic organism can then be out through one or more of any phenotype screen. Suitable general and directed phenotypic screens include the use of fundus photography, blood pressure, behaviour analysis, X-ray fluoroscopy, dual-energy X-ray absorptiometry (DEXA), CAT scans, complete blood counts (CBC), urinalysis, blood chemistry, insulin levels, glucose tolerance, fluorescence-activated cell sorting (FACS), histopathology, expression data, developmental biology. The methodology of the present invention will have broad application in many areas where temporal gene regulation would be advantageous and in validating putative drug targets identified in genomics programmes.

The present invention may be used to modulate the expression of genes that are associated with human disease. A non-exhaustive list of genes is set out below (homologs of the genes are included):

Genes relating to cancer include, but are not limited to, Cdh3, Ncam, Akp2, Asgr2, Bax, Bmp4, Ccnd1, Cd38, Cdc37, Cdkn1a, Cdkn1b, Cdkn1c, Csk, Epas1, Fgf2, Grpr, HBV, Igf1, Inhbb, Inpp5d, IRS1, Itga5, Kcna1, lacz, Map2k4, Mdm2, Nfkbia, Ngfb, Oxt, Pemt, Plp, Shh, Src, Stat5a, Tcfap2a, Trp53, Blmh, Cd152, Cmkar2, Cmkbr5, Csf1, Csf3, Egfr, Gzmb, Ifng, Ifngr, IGFBP3, Il1r1, Il1rap, Il2, Il2ra, Il2rb, Il2rg, Il4, Il4ra, Il5, Il6, Il7r, Il10, Il12a, Il12b, Il12rb1, Il12rb2, IRS1, Kdr, Lifr, Lta, Ncam, Ntf3, Ntf5, Ntrk1, Ntrk2, Ntrk3, Ph, Prlr, Scya3, Smst, Tgfa, Tgfb1, Tgfb2, Tgfb3, Tnf, Tnfrsf1a, Tnfrsf1b, Tnfrsf5, Apc, Prkdc, TAg, Amh, Kit, Kitl, Ter, Fech, hr, Atm, E2f1, Hox11, Apc, Cdh3, Erbb2, Hras, Met, Notch4, PIP, PyVT, Tag, Wnt1, Madh3, Nf1, Ptch, Rb1, Odc, Bcl3, Fos, Fyn, Jun, Kras2, luc, Mos, Myc, Rab3a, Rela, Yes, Cd44, Mgmt, Plg, Ahr, Pgy2, Rag1, Btk, Igh-6, Jak3, Tcra, Tcrb, Tcrd, Ttp53, Ttpa, Vhlh and Wt1.

Genes relating to diabetes and obesity include, but are not limited to, Ins2, Ins1, H2-Ea, H2-Ab1, Ifng, Prkdc, B2m, Rag1, Lep, Lepr, Cpe, Gck, Irs1, Irs2, Irs3, Irs4, Slc2a1, Cre, Dgat, tub, Pcsk2, Insr, Nos1, Nos3, Tnf, B2m, Thy1, Pomc, Ppara and Csf2.

Genes relating to diseases of the cardiovascular system include, but are not limited to, Acact, Alox15, Apoa2, Apob, Apoe, Ath1r, Cdkn1a, Cyp7a1, Epas1, Lcat, Ldlr, Pemt, Soat1, fld, hr, Ace, Adra1b, Adrb2, Adrbk1, Anx6, Atp7a, Cdh2, Evil, Fn1, Gja1, Itga4, Jup, Kif3a, Nf1, Nos3, Nppa, Thra, Vcam1, Wt1, Agt, Bdkrb2, Bmp4, Drd3, Kcna1, Npr3, Ren, Apoc1, Apoc2, Apoc3, Apoa1, Cetp, Hpl, Lipc, Srb1, Adra2a, Agtr1a, Fgf2, Tnf, Asgr2, Lrpap1, Vldlr, Col3a1 and Plg.

Genes relating to diseases of the endocrine system include, but are not limited to, A, Cpe, fld, Insr, Lep, Lepr, tub, Acact, acd, Cacnb4, Crh, Foxn1, gl, Bmp4, Csf1, dwg, fsn, Hcph, Kit, Kitl, Mitf, oc, Phex, Prlr, Sparc, Grpr, Amh, Ar, Cga, Fshb, jsd, Ghrhr, Hmgic, Myo5a, Nr5a1, Oxt, p, Pit1, Prop1, Smst, Agt, Igf1, Gck, Pcsk2, Egfr, Foxn1, Mc1r, Tgfa, Thrb, Tshr and Ttr.

Genes relating to apoptosis include, but are not limited to, Fas, Ngfr, Tnfrsf1a, Tnfrsf1b, Bax, Bcl2, E2f1, Mdm2, Pcc, Rb1, Trp53, Bdnf, Fasl, Gzmb, Ntf3, Ntf5, Pfp, Tag and Tnf.

Genes relating to immunology and inflammation include, but are not limited to, Cd1, Cd3e, Cd3z, Cd4, Cd44, Cd5, Cd8a, Cd8b, Cd14, Cd152, Cd28, Cd38, Fcer1g, Fcgr2a, Fcgr2b, Fcgr3, Gpi1, H2-Aa, H2-DMa, H2-Eb1, H2-Eb2, H2, Hc, Icam1, Igh-1, Igh-5, Igh, Igk-C, Igl-1, Igl-5, Itga4, Itga5, Itgb2, Itgp, Lyst, Mar1, Ncam, PCC, Pep3, Ptprc, Ptprcap, PVR, Sele, Sell, Selp, Spn, Tapbh, Tcra, Tcrb, Tcrd, Thy1, Tlx1, Tnfrsf5, Tnfrsf6, Tnfsf5, Bmp4, Cmkar2, Cmkbr5, Csf1, Csf3, Egfr, Gzmb, Ifng, Ifngr, Il1r1, Il1rap, Il2, Il2ra, Il2rb, Il2rg, Il4, Il4ra, Il5, Il6, Il7r, Il10, Il12a, Il12b, Il12rb1, Il12rb2, Il15ra, Irs1, Itgb7, Kdr, Kitl, Lifr, Lta, Map2k4, Ntf3, Ntf5, Ntrk1, Ntrk3, Ph, Scya3, Smst, Tgfa, Tgfb1, Tgfb2, Tgfb3, Tnf, Tnfrsf1a, Tnfrsf1b, A, Atm, C3, C4, Cacnb4, Cd80, Cd86, Dh, Dsg3, Eef1a2, gl, hr, Lama2, Lbp, Lep, Lepr, Mitf, Pit1, Prop1, Scn8a, Abcb2, Ada, B2m, Bcl2, Bcl3, Btk, C2ta, Foxn1, H2-Ab1, Hcph, Igh-6, Igh-J, Ii, Jak3, Kit, Lck, Ltb, Lyn, Nfkb1, Nfkbia, Pfp, Pnlliprp2, Prkdc, Ptprcap, Rag1, Relb, Stat4, Stat6, Tlr4, Alox5, Alox5ap, Alox15, Bdkrb2, Blmh, Bmp6, Cmo, Crh, Nos2, Ptgs2, Vr1, Bax, E2f1, Inpp5d, Rb1, Stat5a, Trp53, Fyn and Irf1.

Genes relating to neurobiology include, but are not limited to, Apoe, Atm, Bdnf, Cdk5, Chrna7, Cmkar4, Cstb, Gad2, Gfap, Gria2, Grik2, HD, Hdh, Nos1, Nos1, Penk-rs, Prkcc, Psen1, Snca, Tnf and Vr1.

In addition to the therapeutic gene or genes and the expression regulatory elements described, the delivery system may contain additional genetic elements for the efficient or regulated expression of the gene or genes, including promoters/enhancers, translation initiation signals, internal ribosome entry sites (IRES), splicing and polyadenylation signals. Expression levels may be improved by incorporating elements such as the WPRE.

The delivery of one or more one or more therapeutic genes by a delivery system according to the present invention may be used alone or in combination with other treatments or components of the treatment. In a further preferred embodiment of the first aspect of the invention, one or more nucleotides of interest (NOI) is introduced into the vector at the cloning site. Such therapeutic genes may be expressed from a promoter placed in the retroviral LTR or may be expressed from an internal promoter introduced at the cloning site.

For example, the delivery system of the present invention may be used to deliver one or more NOI(s) useful in the treatment of the disorders listed in WO98/05635. For ease of reference, part of that list is now provided: cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the delivery system of the present invention may be used to deliver one or more NOI(s) useful in the treatment of disorders listed in WO98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the delivery system of the present invention may be used to deliver one or more NOI(s) useful in the treatment of disorders listed in WO98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The subject treated by the method of the present invention may be an animal subject. Preferably the subject is a mammalian subject, more preferably a human subject.

The present invention also provides a pharmaceutical composition for treating an individual by gene therapy, wherein the composition comprises a therapeutically effective amount of the delivery system of the present invention and optionally comprising one or more deliverable therapeutic and/or diagnostic NOI(s). Since the delivery system is a viral delivery system then the composition may in addition or in the alternative comprise a viral particle produced by or obtained from same. The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The delivery of one or more therapeutic genes by a delivery system according to the invention may be used alone or in combination with other treatments or components of the treatment.

The non-primate lentiviral vector particles of the present invention are typically generated in a suitable producer cell. Producer cells are generally mammalian cells but can be for example insect cells. A producer cell may be a packaging cell containing the virus structural genes, normally integrated into its genome. The packaging cell is then transfected with a nucleic acid encoding the vector genome, for the production of infective, replication defective vector particles. Alternatively the producer cell may be co-transfected with nucleic acid sequences encoding the vector genome and the structural components, and/or with the nucleic acid sequences present on one or more expression vectors such as plasmids, adenovirus vectors, herpes viral vectors or any method known to deliver functional DNA into target cells.

The vectors of the invention, for example, the lentiviral vectors of the first aspect of the invention, may be used to deliver an NOI to any prenatal cell. The term “prenatal” means occurring or present before birth. In one embodiment the method is applied to a cell at the embryonic stage. The term embryo includes animals in the early stages of development up to birth (or hatching). As used herein the term “embryo” includes “pre-embryo”, i.e. the structure formed after fertilisation of an ovum but before differentiation of embryonic tissue, and includes a zygote and blastocyte. The term also includes a fetal cell, i.e. an embryonic cell which is in the latter stages of development. The present invention also encompasses delivery to a perinatal cell. The term “perinatal” refers to the period from about 3 months before to about one month after birth, and includes the neonatal period. The term “neonate” refers to the first few weeks following birth.

Generally vectors of the invention, for example, the lentiviral vectors of the first aspect of the invention may be used to deliver an NOI to any germ cell, including a primordial germ cell, or cell which is capable of giving rise to a germ line change. The term “germ cell” is the collective term for cells in the reproductive organs of multicellular organisms that divide by meiosis to produce gametes. The term “gametes” refers to the haploid reproductive cells—in effect the ovum and sperm. However, as indicated above the present invention is also applicable to cells involved in gametogenesis and cells from structures in which gametogenesis take place, such as the ovary.

Gametogenesis will now be described in relation to mammals by way of example only. Vectors such as the lentiviral vector may be used to deliver an NOI to any of the cells of structures mentioned below. It will be appreciated that the equivalent processes in non-mammalian organisms are also included in the present invention. In brief, gametogenesis is the process of forming gametes (by definition haploid, n) from diploid cells of the germ line. Spermatogenesis is the process of forming sperm cells by meiosis (in animals, by mitosis in plants) in specialized organs known as gonads (in males these are termed testes). After division the cells undergo differentiation to become sperm cells. Oogenesis is the process of forming an ovum (egg) by meiosis (in animals, by mitosis in the gametophyte in plants) in specialized gonads known as ovaries.

In spermatogenesis the sperms are formed from the male germ cells, spermatogonia, which line the inner wall of the seminiferous tubules in the testis. A single spermatogonium divides by mitosis to form the primary spermatocyte, each of which undergoes the initial division of meiosis to form two secondary permatocytes. Each of these then undergoes a second meiotic division to form two spermatids, which mature into spermatozoa. The testis is composed of numerous seminiferous tubules, in whose walls spermatogenesis takes place. The primordial germ cells are formed in the germinal epithelium lining towards the outside of the tubule, and as cell divisions proceed the daughter cells move towards the lumen of the tubule. All these cells are nourished and supported by neighbouring Sertoli cells.

In oogenesis a primary oocyte is formed by differentiation of an oogonium and then undergoes the first division of meiosis to form a polar body and a secondary oocyte. Following fertilisation of the egg, the secondary oocyte undergoes the second meiotic division to form the mature ovum and a second polar body. The ovary contains many follicles composed of a developing egg surrounded by an outer layer of follicle cells. After ovulation the egg moves down the oviduct to the uterus.

It will be appreciated that the vector may be administered at one locality, but the NOI is expressed or its effects felt, in another cell of the organism, i.e. the site of administration may be different from the target cell. Cells into which the non-primate lentiviral vector may be administered include the examples of target cells listed above.

More preferably, the cell is at the embryonic stage, and for example is in utero, the lentiviral vector may be administered via the umbilical cord, placenta, or amniotic fluid, or by the intraperitoneal or intrahepatic routes. The introduction of the lentiviral vector is aided by the use of ultrasound.

The production of transgenic animals, using ES cells and otherwise, is well known in the art, and described for example in Manipulating the Mouse Embryo, 2nd Ed., by B. Hogan, R. Beddington, F. Costantini, and E. Lacy. Cold Spring Harbor Laboratory Press, 1994; Transgenic Animal Technology, edited by C. Pinkert. Academic Press, Inc., 1994; Gene Targeting: A Practical Approach, edited by A. L. Joyner. Oxford University Press, 1995; Strategies in Transgenic Animal Science, edited by G. M. Monastersky and J. M. Robl. ASM Press, 1995; and Mouse Genetics: Concepts and Applications, by Lee M. Silver, Oxford University Press, 1995. A useful general textbook on this subject is Houdebine, Transgenic animals—Generation and Use (Harwood Academic, 1997)—an extensive review of the techniques used to generate transgenic animals from fish to mice and cows.

Thus, for example, the present invention permits the introduction of heterologous DNA into, for example, fertilised mammalian ova by lentiviral infection. In one embodiment the fertilised egg is collected from a donor mother at the one cell stage and the transduced cell is transferred to a foster mother. Integration which occurs at the one cell stage produces an organism which is a true transgenic, i.e. transgenic throughout, including the germ cells. If integration occurs at a later stage mosaics are produced. In a highly preferred method, developing embryos are infected with a lentivirus containing the desired DNA, and transgenic animals produced from the infected embryo. Traditional transgenic methods have required that the embryonic cells are transformed ex vivo then reimplanted into the uterus. A significant advantage associated with the present invention is that the NOI can be introduced in utero. Another method which may be used to produce a transgenic animal involves introducing a nucleic acid into pro-nuclear stage eggs by lentiviral infection. Injected eggs are then cultured before transfer into the oviducts of pseudopregnant recipients.

By way of a specific example for the construction of transgenic mammals, such as cows, nucleotide constructs comprising a sequence encoding a therapeutic protein are introduced using the method of the present invention into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, such as cattle, by administering coprostanol. Oestrous is produced within two days and the embryos are transferred to the recipients 5-7 days after estrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

Analysis of animals which may contain transgenic sequences would typically be performed by either PCR or Southern blot analysis following standard methods. If desired, the organism can be bred to homozygosity.

The use of the present invention to produce transgenic organism for use in gene therapy and in the production of disease models has been mentioned above. In particular, disease models allow experimental investigation of gene function. In general transgenic organisms expressing novel genes or genes with a heterologous promoter represent gain-of-function mutations. Loss-of-function mutations can be created by gene targeting to create so-called “knockout” organisms. Transgenic organisms are also useful for the investigation of control regions and expression patterns. Transgenic organisms can also be used to identify novel genes using techniques such as insertional mutation, gene traps and promoter traps. Transgenic animals also have agricultural applications, for example to bring genetic improvements to milk yield, body mass, milk composition, disease resistance etc. Transgenic animals are also useful in so-called pharmaceutical farming in which transgenic livestock are used a bioreactors for the production of therapeutic proteins.

By way of example, the regulated ablation of SMN (homozygous deletion of which results in pre-natal mortality) would provide a useful model of spinal muscular atrophy for gene therapy studies. A CFTR deficiency model is also a valuable application. Other putative candidates include: presenilin-1, RARα, BDNF, VEGF and EGFR.

The analysis of resultant phenotypes can be carried out using standard techniques such as histological tissue analysis and microarray gene expression profiling.

In accordance with a preferred feature of the present invention a lentiviral vector, and preferably an EIAV vector, is used to produce transgenic chickens that produce therapeutic or diagnostic proteins in their eggs.

The use of lentiviral vectors to produce transgenic avians allows the expression of genes throughout significant numbers of generations with-out the foreign gene silencing observed with some retroviral vectors. For example Mizuarai et al. (2001) (Biochemical and Biophysics Research Communications; 286: 456-463) observed that LTR driven expression from MLV based vectors in transgenic quail could not be observed, whilst expression from an internal Rous sarcoma virus (RSV) promoter was present in G₀, G₁ and G₂ birds.

A lentiviral vector encoding for a therapeutic protein or a protein of diagnostic use, for example an antibody or a fragment of an antibody, may be used to produce transgenic chickens. The antibody may be engineered to contain domains derived from more than one animal species or to contain domains that bind to different target molecules. The nucleotide coding sequence of the target gene can be altered to increase RNA stability or RNA transcription levels without altering the amino acid sequence of the resultant protein.

Expression of the therapeutic/diagnostic gene may be from a constitutive promoter or from a promoter that confers tissue specific expression. For instance expression of the target protein may be restricted to the reproductive structures (including the oviduct or reproductive tract) in such a way as to result in the target protein being present in eggs. Promoters or elements from promoters of genes for proteins found in egg white such as the ovalbumin, lysozyme, conalbumin and ovomucoid may be used. The expression of these genes is regulated by the steroid hormones but there is evidence for the ovalbumin and conalbumin promoters that other cell specific transcription factors are also involved (Dierich et al EMBO J. 1987 August; 6(8):2305-12). The ovalbumin gene promoter has been shown to have tissue specific silencing elements between −3200 and −2800 bp from the transcription site (Muramatsu et al. Mol Cell Biochem 1998 August; 185(1-2):27-32), whereas a silencing element is present −2400 bp from the transcription site of the lysozyme gene (Bonifer et al. J Biol Chem. 1997 Oct. 17; 272(42):26075-8. Review). However Dierich et al. (1987) obtained some degree of cell specificity in a truncated ovalbumin extending from −1348 to −1. Some degree of steroid regulation was observed for a truncated ovalbumin extending from −425 to −1 in primary cultured chicken oviduct tubular gland cells (Dierich et al. 1987).

Lentiviral vectors encoding for therapeutic/diagnostic proteins are used to transduce cells in the blastoderm stage embryo in new-laid eggs by injection. Alternatively, lentiviral vectors can be used to transduce earlier stage embryos using techniques such as those described in WO 90/13626 or similar published techniques to allow the embryo to develop normally.

In brief a uterine embryo is abstracted from a hen either manually or by inducing premature oviposition. The embryo is transduced with the lentiviral vector and then cultured to fruition. This allows cells of the embryo to be transduced whilst the number of cells present is relatively low and increases the number of birds produced in which the introduced gene is present in the germ line and is inherited.

The present invention also relates to the use of RNAi to enhance protein yield from transgenic avians

In order to maximise the expression of a desired protein in eggs, as described in example 8 below, RNAi may be used to decrease the proportion of abundant proteins normally present in the egg white such as ovalbumin, lysozyme, conalbumin and ovomucoid. Down-regulating the production of these proteins may result in a concomitant increase in the proportion of the desired protein in the egg giving improved yields.

This may be achieved by expressing siRNAs, or short hairpin pre-cursors, targeting the required mRNA from an EIAV vector. The vector may comprise siRNAs targeting one or more mRNAs at one or more sites within the mRNA.

Efficient targeting of proteins such as ovalbumin may result in eggs which are non-viable for breeding purposes. Transgenic roosters may therefore be identified and crossed to hens transgenic for a vector containing the transgene of interest in order to obtain female offspring with both traits which would be used as bioreactors for protein production. Regulation of the siRNAs (as described in example 10) would be an alternative to this: silencing may be induced in laying hens when eggs are required for protein production, but not for breeding. This would also overcome any possible deleterious effects of down-regulating expression of the target genes within the whole organism.

The present invention will now be described by way of further example with reference to the following non-limiting Examples:

### EXAMPLE 1

### EIAV Transduction of Perinatal Animals

### Preparation of VECTOR

Vector was prepared by transient co-transfection of 293T human embryonic kidney cells as previously described (Mitrophanous et al 1999). The EIAV vector genome, SMART2Z, expresses the β-galactosidase reporter gene from an internal CMV promoter. It contains the EIAV central polypurine tract (cPPT) (Stetor et al 1999) and the Woodchuck Hepatitis Post-Transcriptional Regulatory Element (WPRE) (Donello et al 1998).

### Administration of Vector

Vector was administered by injecting foetuses intra-vascularly as follows:

Under isoflurane anaesthesia a full depth midline laporotomy was performed to expose the uteri of pregnant mice at 16 days gestation. For each foetus 2×10⁷ T.U. (transforming units) of vector was administered in a total volume of 20 μl, using a 34-gauge needle (Hamilton), into a peripheral yolk sac vessel. Up to five foetuses were injected per dam. The laporotomy was closed by suturing layer to layer and mice allowed to recover in a warm cage.

### Detection of Gene Transfer

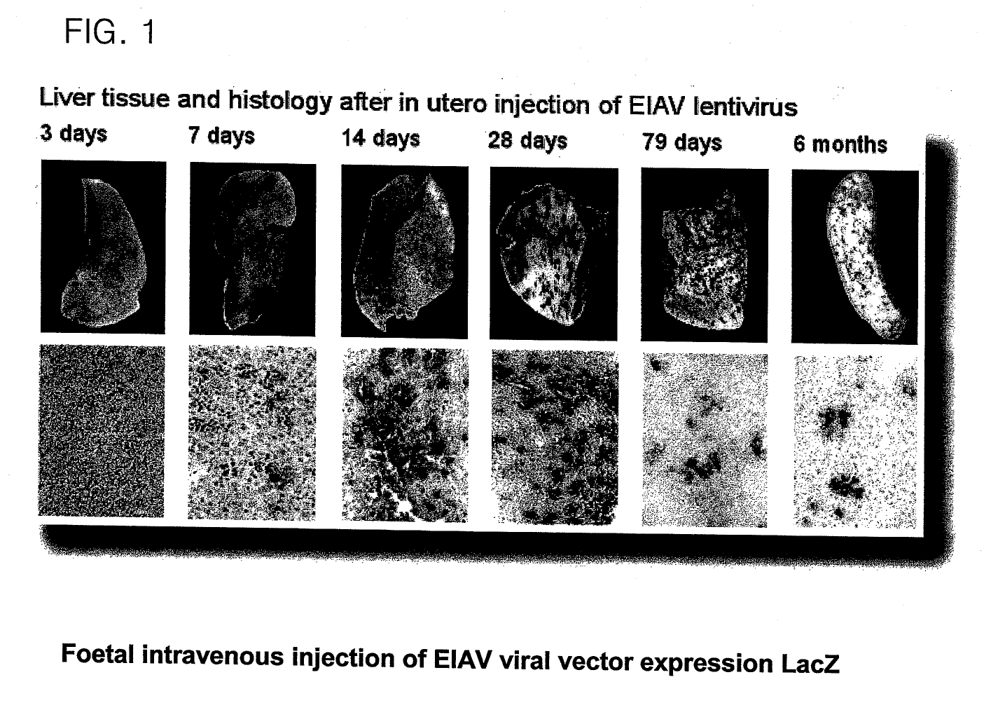

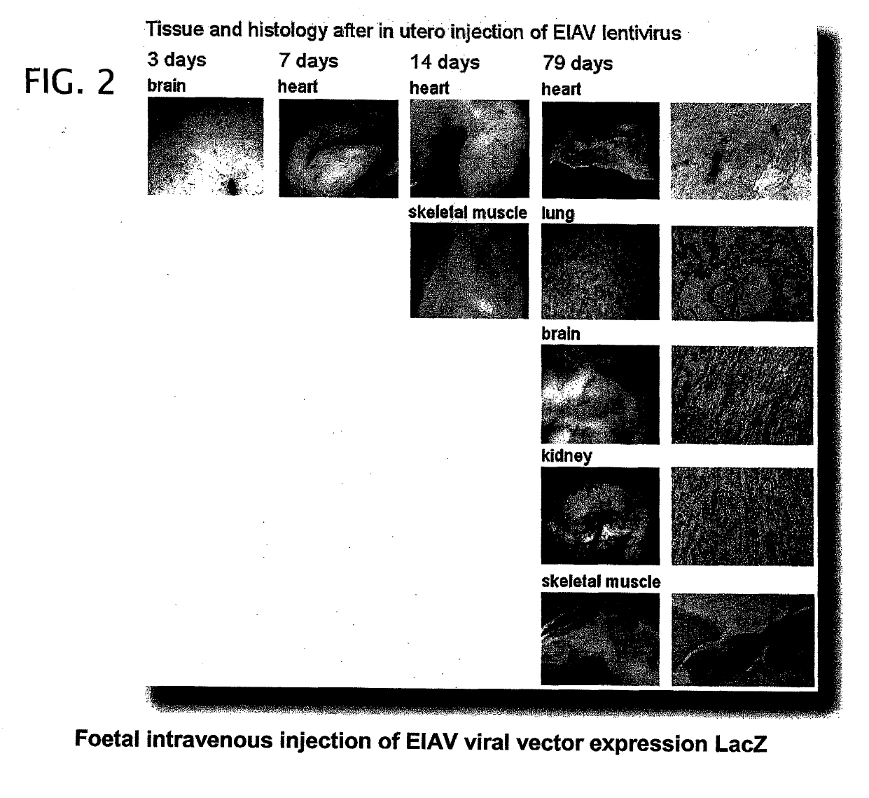

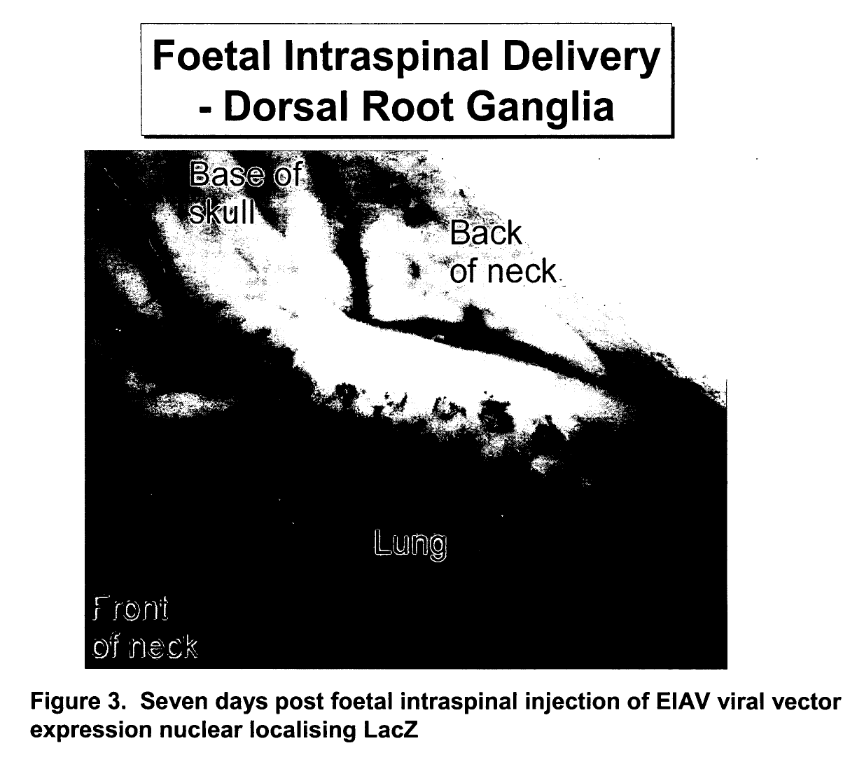

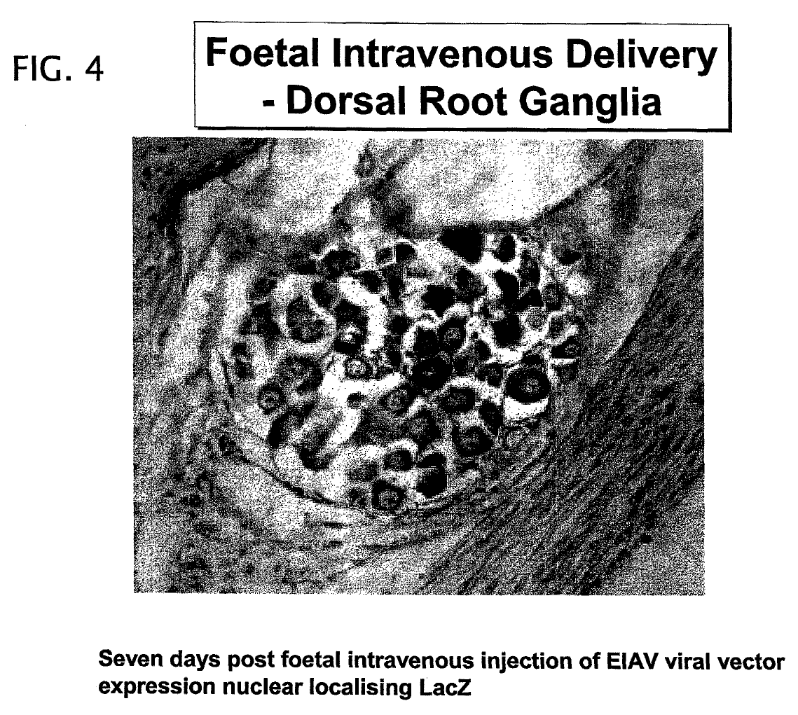

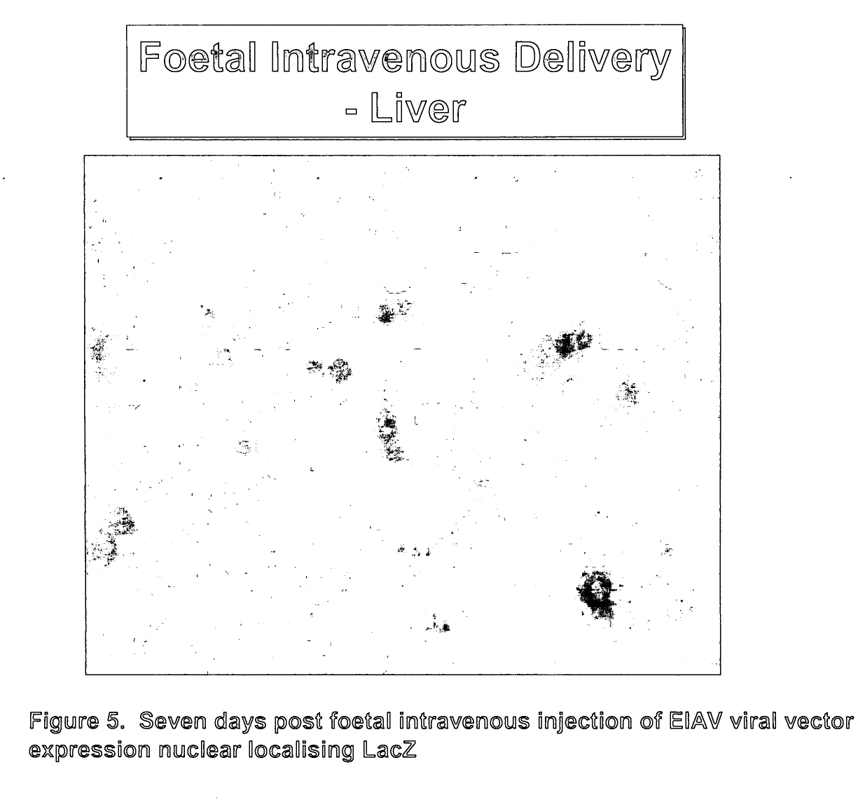

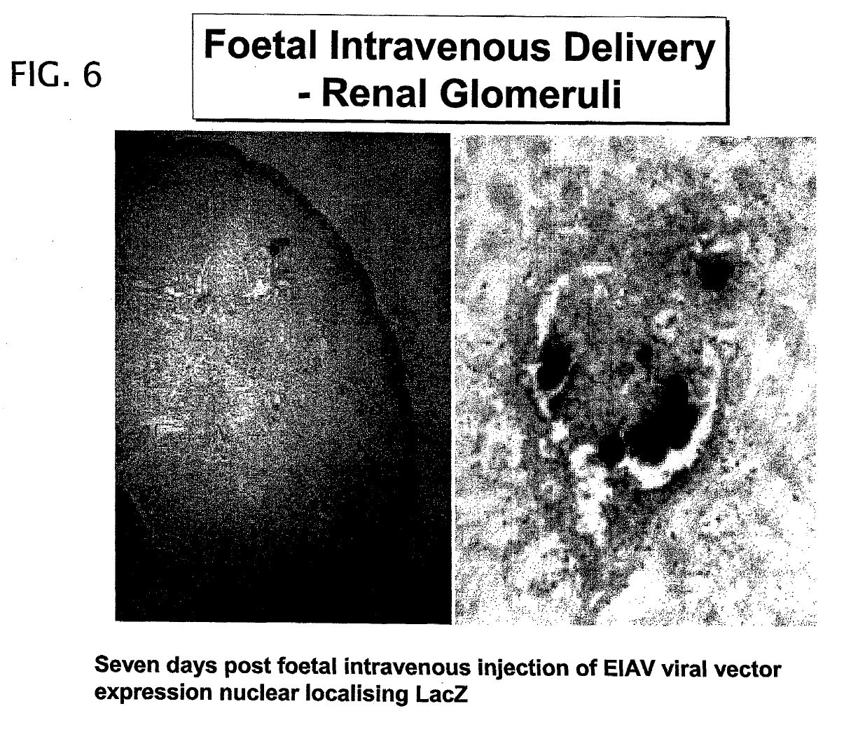

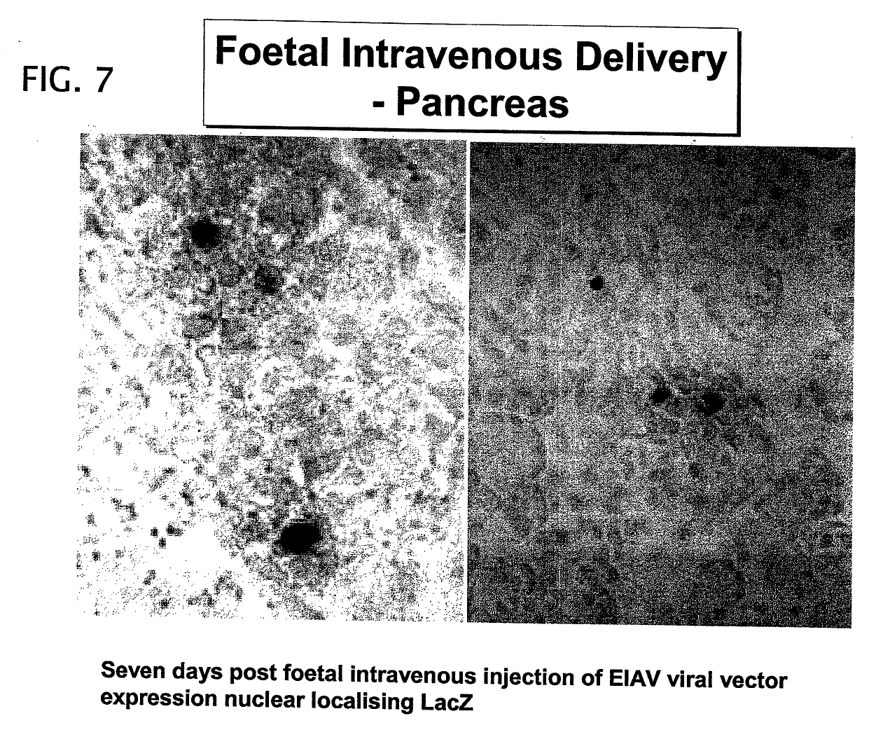

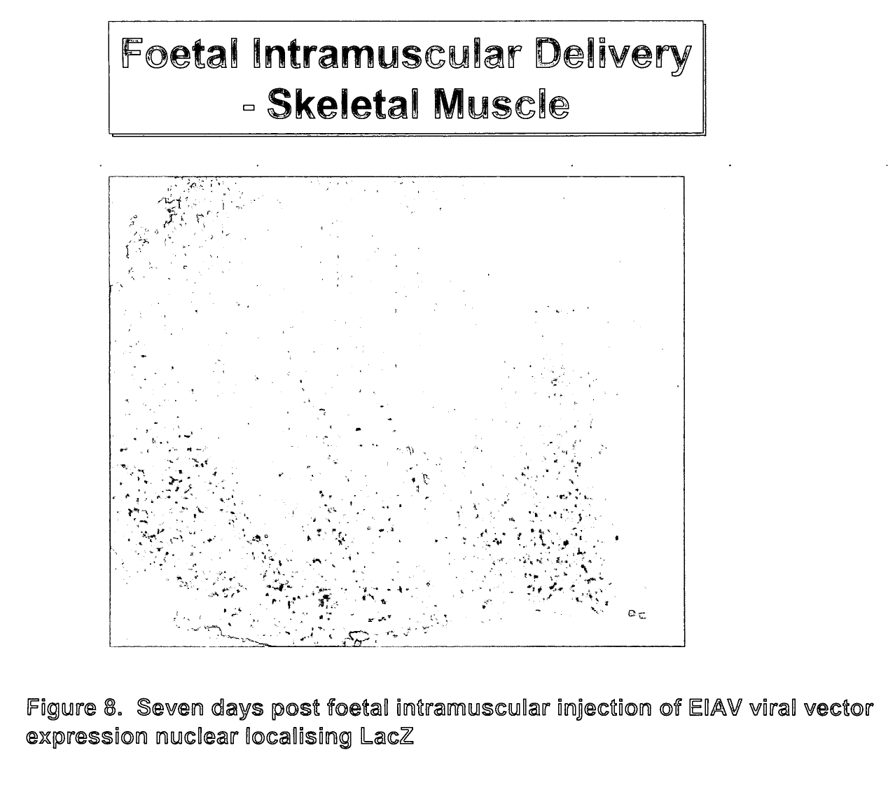

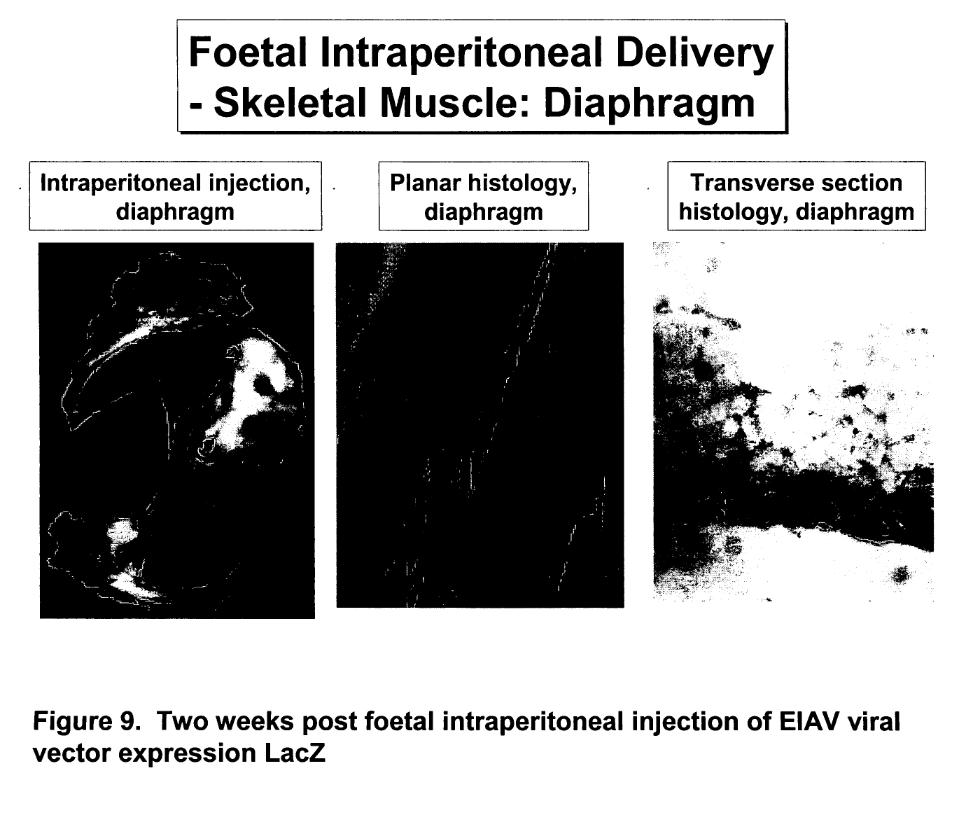

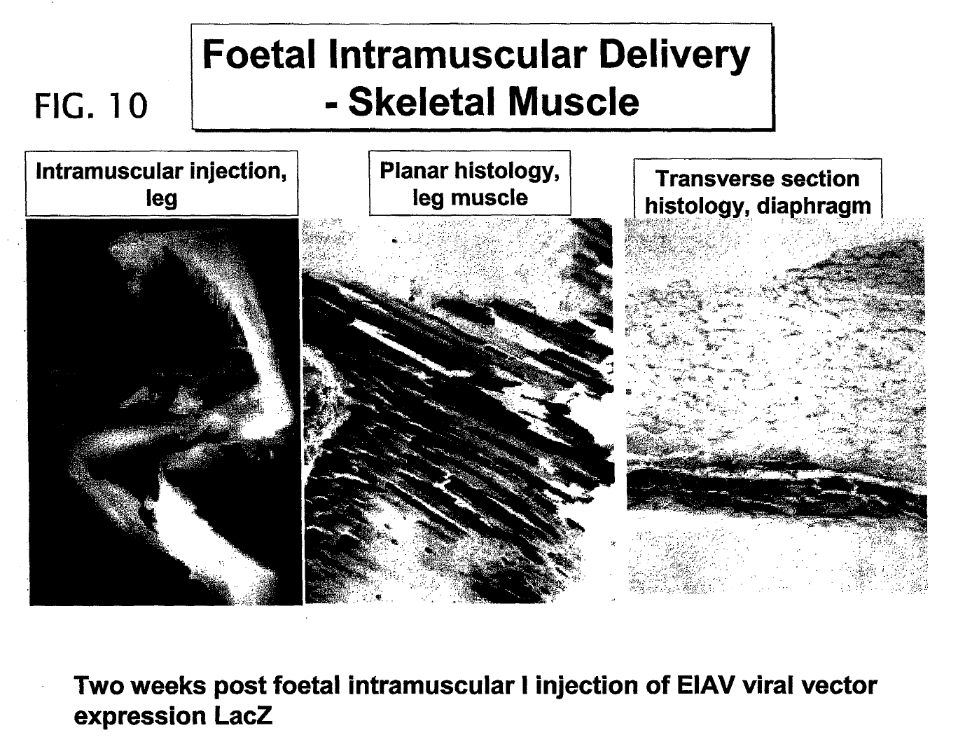

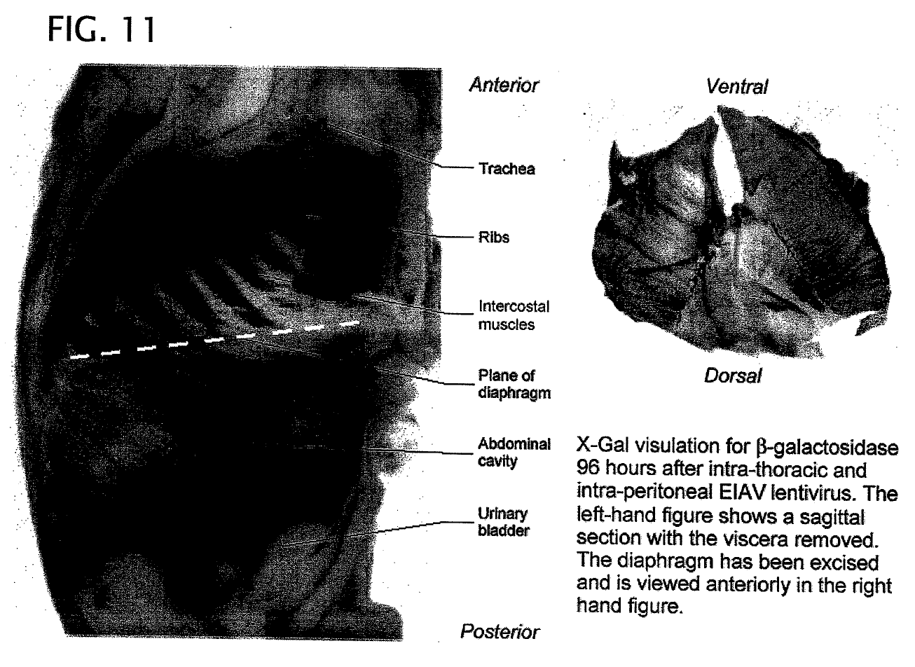

Pups were born at 18-19 days after conception. Mice were sacrificed at various stages of development (3, 7, 14, 28 and 79 days post-injection) by euthanising with isoflurane anaesthesia and samples prepared for histology. Organs were placed in 100% ethanol solution for 2 h prior to staining with X-gal solution (1 mg/ml 5-bromo-4-chloro-3-indolyl-β-D-galactopyranoside dissolved in dimethylsulphoxide, 5 mM potassium ferricyanide, 5 mM potassium ferrocyanide, 2 mM magnesium chloride) for the β-galactosidase marker gene expressed by the vector. Stained tissue was fixed in 10% formaldehyde for 2 h and paraffin embedded, sectioned and counterstained with neutral red. Staining showed transduction of a number of organs including liver, lung, heart, muscle, kidney, skeletal muscle and brain. The results are shown in FIGS. 1 to 11.

Expression levels did not decrease over the period of the study and clonal expansion of transduced cells was observed.

In addition to injection into the yolk sac vessel or umbilical vein, injection directly into the circulation, CSF or other tissue may be carried out, or into the amniotic fluid. The latter may be particularly appropriate when transduction of lung or skin tissue is desired.

### EXAMPLE 2

### Haemophilia

This Example is carried out following the methodology of Example 1. Haemophilia is a blood condition in which an essential clotting factor is either partly or completely missing. It is an X-linked recessive disorder. There are two types of haemophilia, the most common being haemophilia A, in which Factor VIII is lacking. In haemophilia B, Factor IX is lacking. EIAV is used to deliver factor VIII or IX by EIAV to the umbilical vein of haemophiliac foetus or hepatic portal vein of perinates.

### Preparation of the Vector

A vector such as those described in our co-pending GB0202403.1. In more detail:

pONY 8.4 series of vectors has a number of modifications which enable it to function as part of a transient or stable vector system totally independent of accessory proteins, with no detrimental effect on titre. Conventionally lentiviral vector genomes have required the presence of the viral protein rev in producer cells (transient or stable) in order to obtain adequate titres. This includes current HIV vector systems as well as earlier EIAV vectors.

There are 3 modifications when compared with the pONY 8.1 series of vector genomes, these are:
a) All the ATG motifs which are derived from gag and form part of the packaging signal have been modified to read ATTG. This allows the insertion of an open reading frame which can be driven by a promoter in the LTR.
b) The length of the genome i.e. distance between the R regions is closer to that seen in the wt virus (7.9 kb).
c) The 3′ U3 region has been modified to include sequences from the moloney leukemia virus (MLV) U3 region, so upon transduction it can drive second open reading frame (ORF) in addition to the internal cassette, In this example we have MLV but this could be any promoter.

Further details on modifying LTRs can be found in our WO96/37623 and WO98/17816.

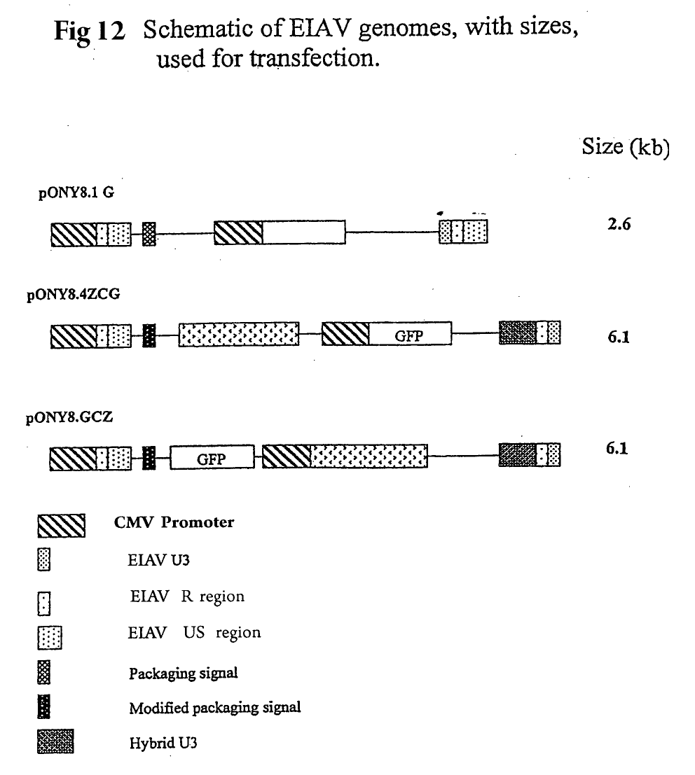

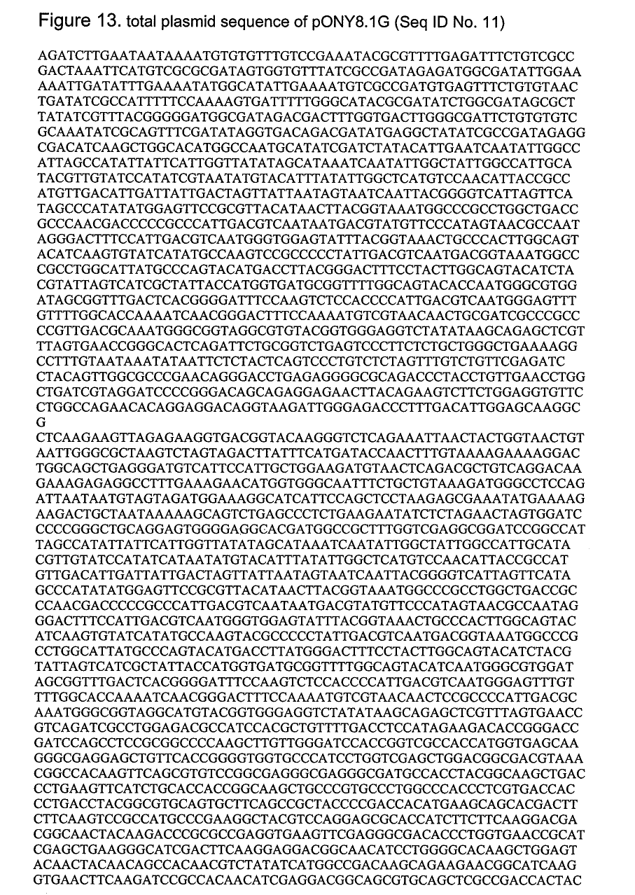

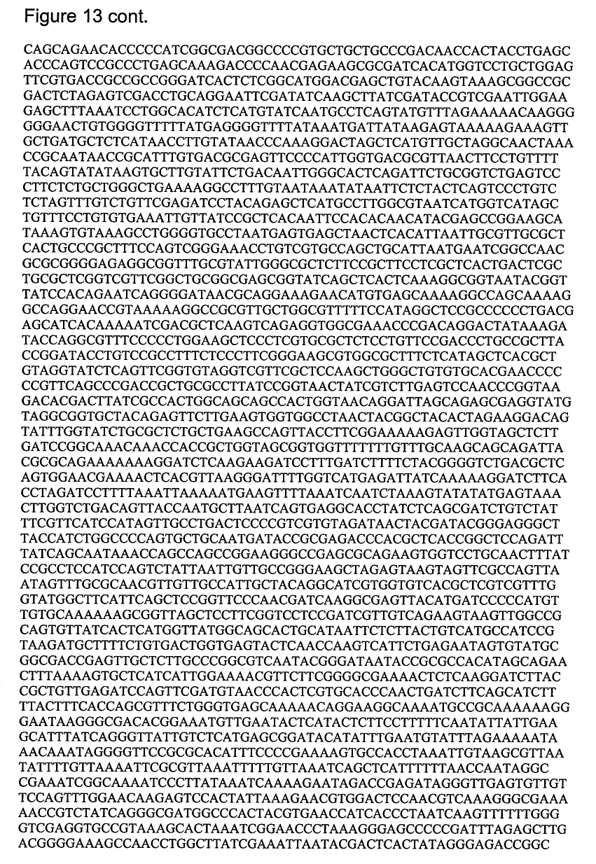

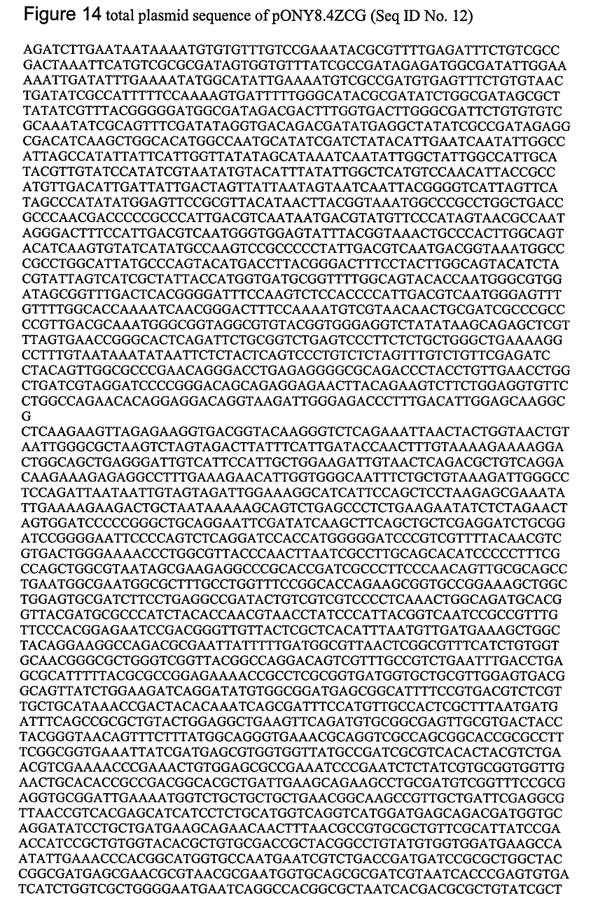

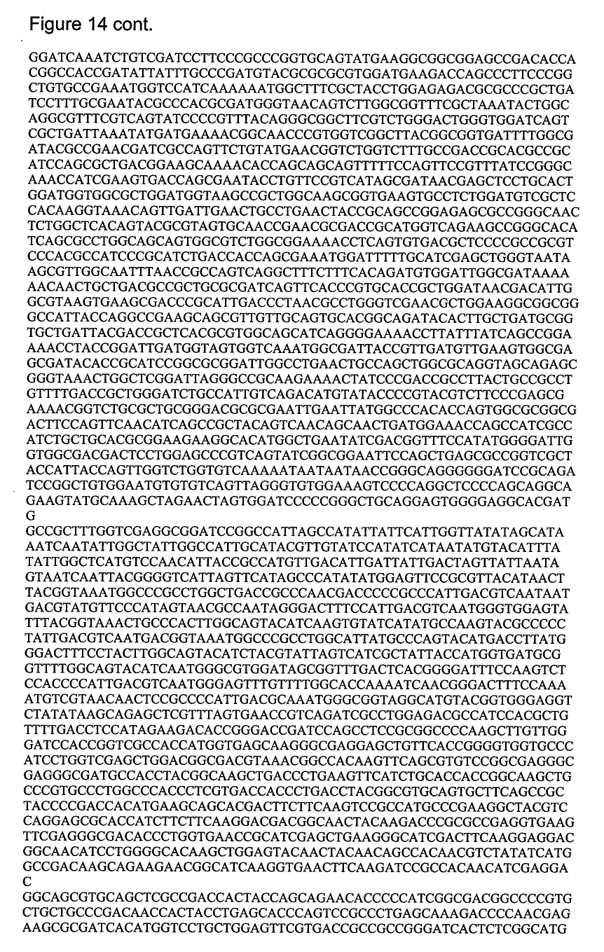

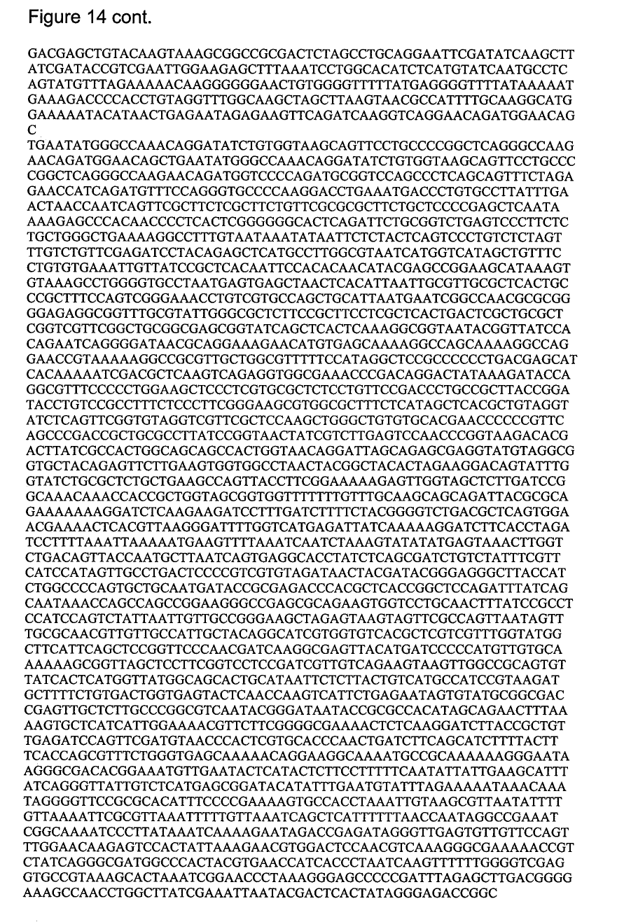

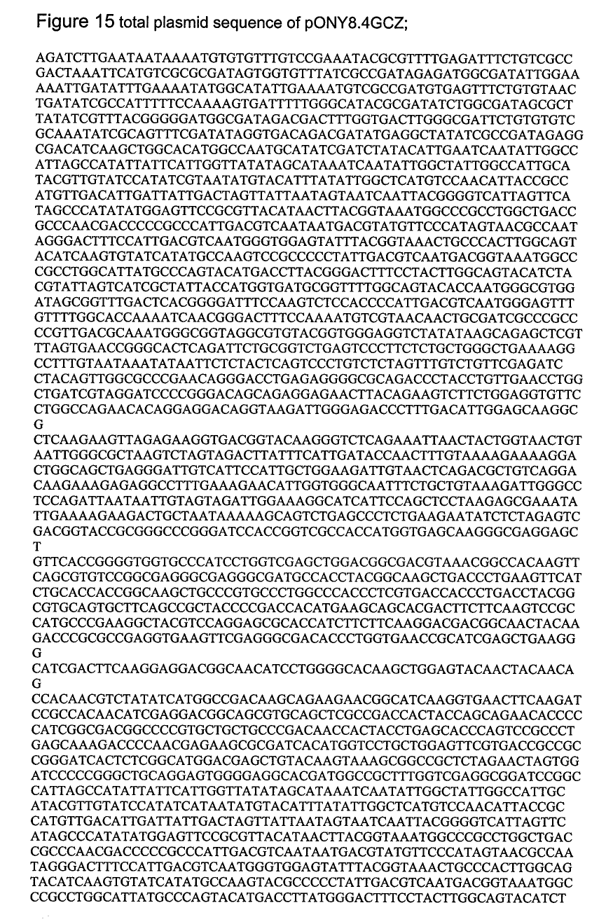

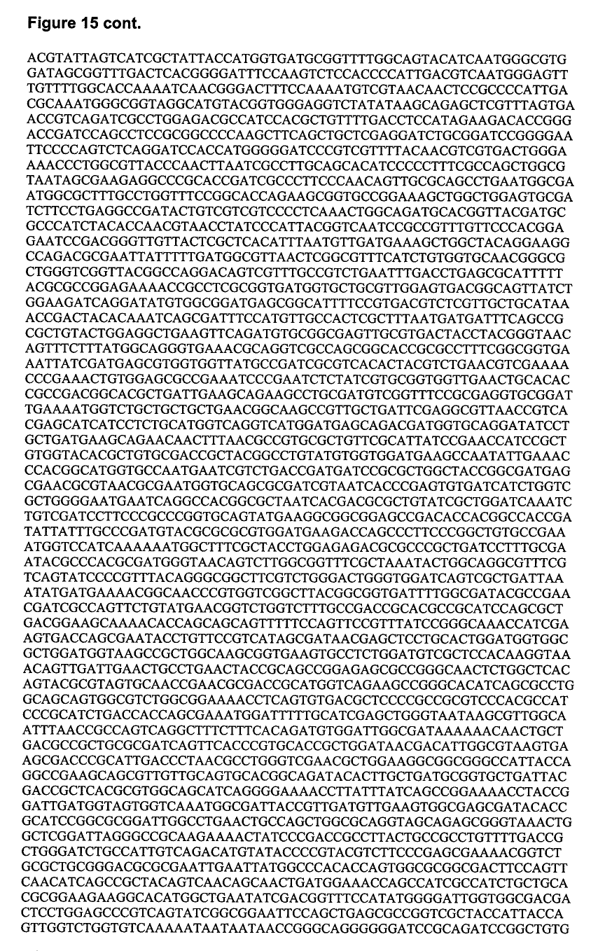

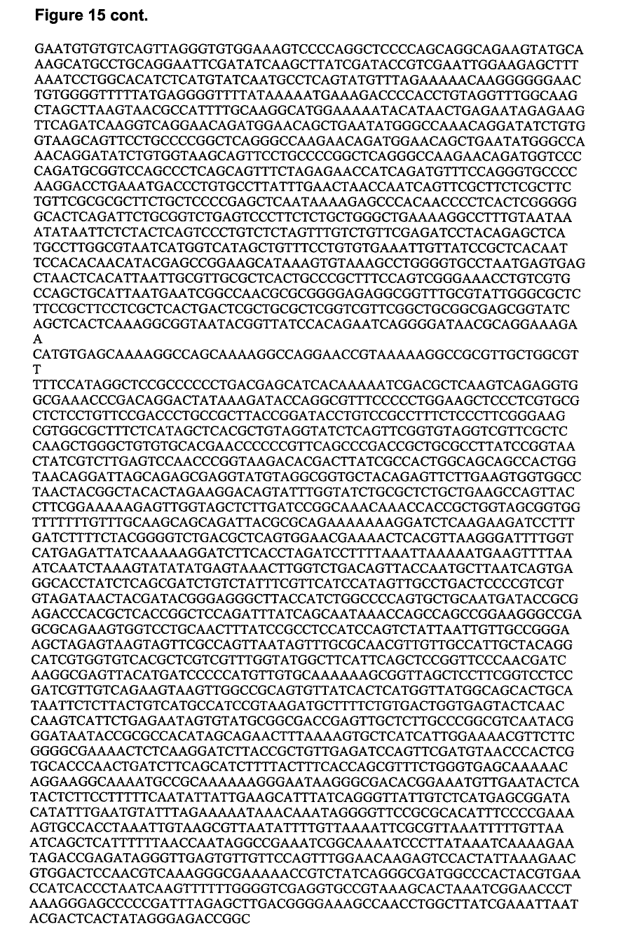

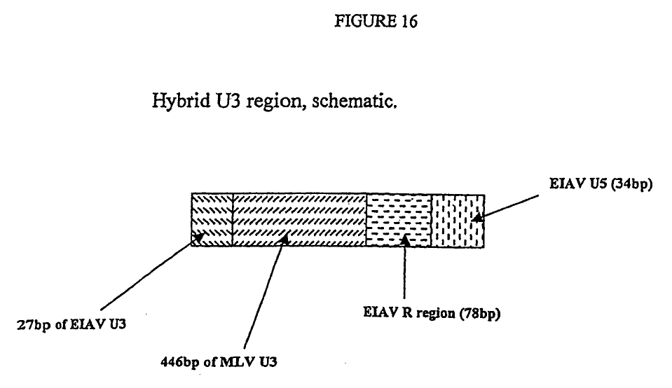

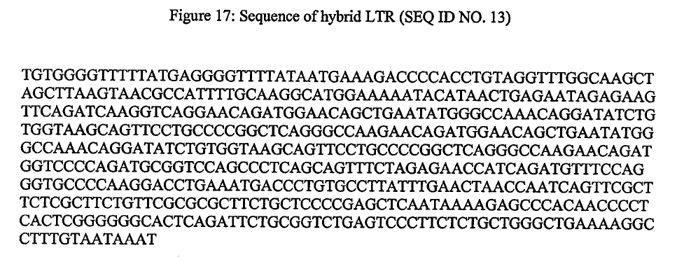

FIG. 12 is a schematic representation of EIAV genomes. These may be used for transfection in accordance with the method of the present invention. Upon transfection the 3′ LTR will be copied to the 5′ LTR. FIG. 13 gives the total plasmid sequence of pONY8.1G. FIG. 14 gives the total plasmid sequence of pONY8.4ZCG. FIG. 15 gives the total plasmid sequence of pONY8.4GCZ. FIG. 16 is a schematic representation of the hybrid U3 region. FIG. 17 gives the sequence of the hybrid LTR.

### The Construction of EIAV/MLV Hybrid LTR Vectors

PCR was carried out as follows:

Product A=primers KM001+KM003, with the pONY8.1Z as target.

Product B=primers KM004+KM005, with the pHIT111 as target.

Product C=primers KM006+KM002, with the pONY8.1Z as target.

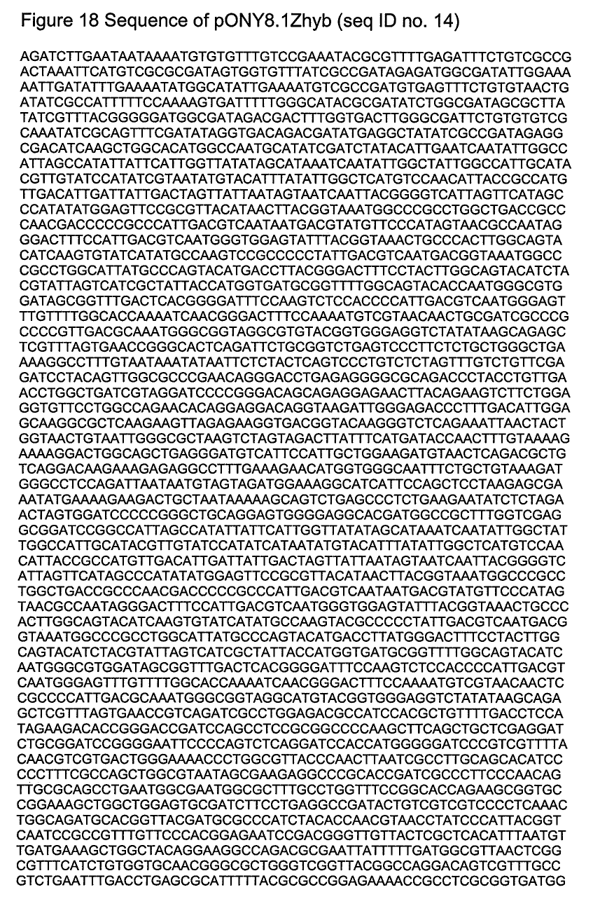

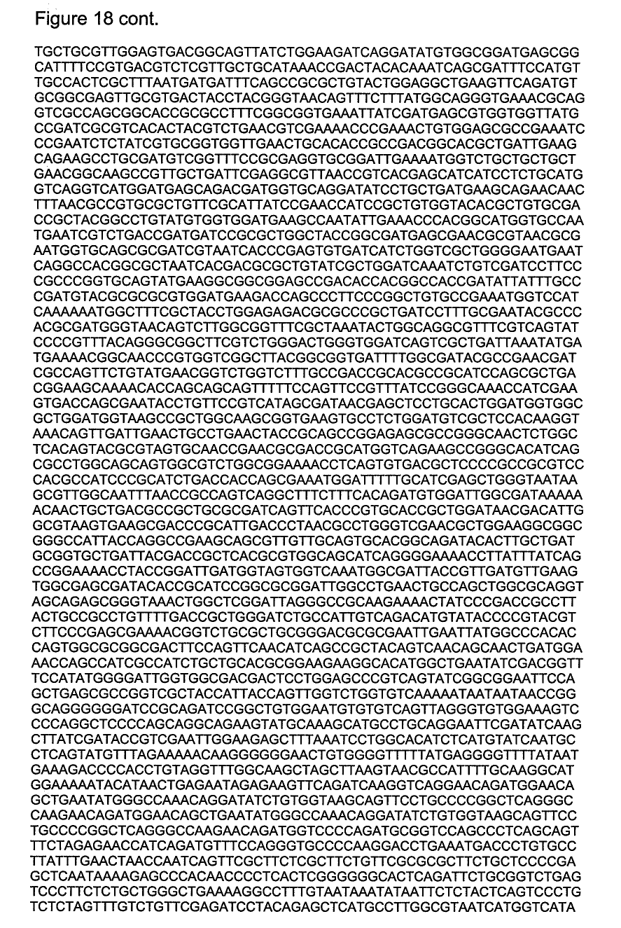

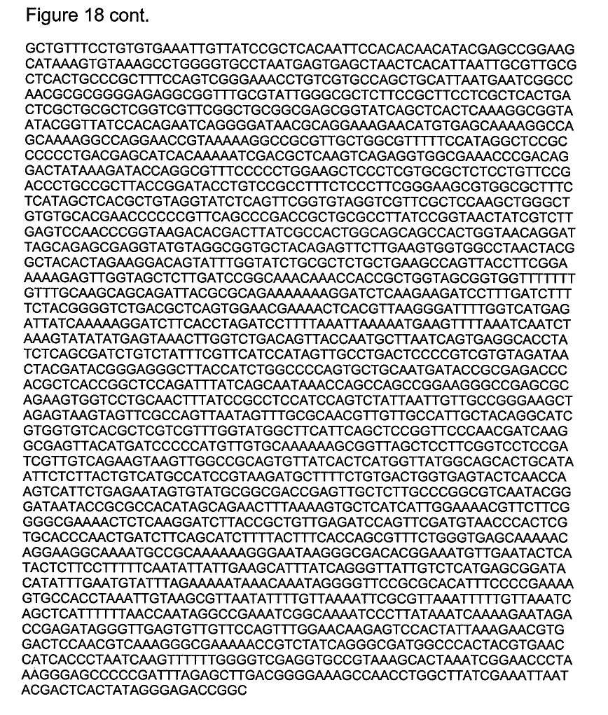

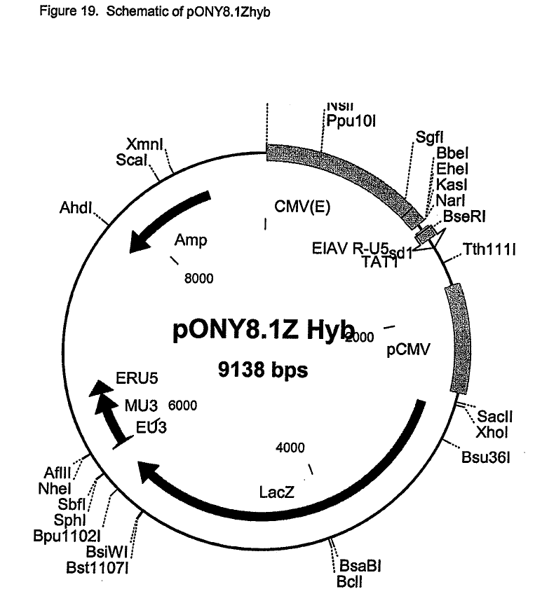

The PCR products (A, B and C) were gel purified. A PCR reaction was set up using Product A and B (with primers KM001 and KM005) to give Product D. A PCR reaction was set up using Product B and C (with primers KM004 and KM002) to give Product E. Product D and E were gel purified and used in a PCR reaction, as targets with primers KM001 and KM002 to give Product F. The PCR Product F was gel purified (approximately 1 kb). This was then cut with Sap I and subcloned into pONY8.1Z cut with Sap I. This gave the vector pONY8.1Zhyb shown in FIGS. 18 and 19. The 3′ LTR of EIAV has now been replaced with an EIAV/MLV hybrid LTR. The EIAV U3 has been almost replaced with the MLV U3 region. The EIAV 5′ U3 sequences of the 3′LTR have been retained as these comprise the att site, that is the sequences needed for integration.

The primer sequences are shown below:

EIAV/MLV hybrid U3
KM001
CAAAGCATGCCTGCAGGAATTCG
KM002
GAGCGCAGCGAGTCAGTGAGCGAG
KM003
GCCAAACCTAGAGGTGGGGTGTTTCATTATAAAACCCCTCATAAAAACCC
CACAG
KM004
CTGTGGGGTTTTTATGAGGGGTTTTATAATGAAAGACCCCACCTGTAGGT
TTGGC
KM005
GAAGGGACTCAGACCGCAGAATCTGAGTGCCCCCCGAGTGAGGGGTTGTG
GGCTCT
KM006
AGAGCCCACAACCCCTCACTCGGGGGGCACTCAGATTCTGCGGTCTGAGT
CCCTTC
Sequence of final PCR product.
EIAV PPT/U3
CAAAGCATGCCTGCAGGAATTCGATATCAAGCTTATCGATACCGTCGAAT
TGGAAGAGCTTTAAATCCTGGCACATCTCATGTATCAATGCCTCAGTATG
TTTAGAAAAACAAGGGGGGAACTGTGGGGTTTTTATGAGGGGTTTTATAA
MLV U3
TGAAAGACCCCACCTGTAGGTTTGGCAAGCTAGCTTAAGTAACGCCATTT
TGCAAGGCATGGAAAAATACATAACTGAGAATAGAGAAGTTCAGATCAAG
GTCAGGAACAGATGGAACAGCTGAATATGGGCCAAACAGGATATCTGTGG
TAAGCAGTTCGTGCCCCGGCTCAGGGCCAAGAACAGATGGAACAGCTGAA
TATGGGCCAAACAGGATATCTGTGGTAAGCAGTTCCTGCCCCGGCTCAGG
GCCAAGAACAGATGGTCCCCAGATGCGGTCCAGCCCTCAGGAGTTTCTAG
AGAACCATCAGATGTTTCCAGGGTGCCCCAAGGACCTGAAATGACCCTGT
GCCTTATTTGAACTAACCAATCAGTTCGCTTCTCGCTTCTGTTCGCGCGC
TTCTGCTCCCCGAGCTCAATAAAAGAGCCCACAACCCCTCACTCGGG
MLV U3/EI4V R/U5
GGGCACTCAGATTCTGCGGTCTGAGTCCCTTCTCTGCTGGGCTGAAAAGG
CCTTTGTAATAAATATAATTCTCTACTCAGTCCCTGTCTGTAGTTTGTCT
GTTCGAGATCCTACAGAGCTCATGCCTTGGCGTAATCATGGTCATAGCTG
TTTCCTGTGTGAAATTGTTATCCGCTCACAATTCCACACAACATACGAGC
CGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCG
TGCCAGCTGCATTAATGAATCGGCCAACGCGCGGGGAGAGGCGGTTTGCG
TATTGGGCGCTCTTCCGCTTCCTCGCTCACTGACTCGCTGCGCTC

### Haemophilia A

An EIAV viral vector (such as those described above) expressing Factor VIII (the wild type full length open reading frame (ORF) or a truncated B domain deleted wild type ORF or a codon optimised ORF or a truncated B domain deleted codon optimised ORF) is administered following the methodology in Example 1 and including either intravenous or intra hepatic or intramuscular delivery. A suitable promoter such as CMV or human promoter/enhancers such as PGK is used to express the gene. In addition inducible promoters such as the Tet system can be used to regulate the expression. As an alternative tissue specific promoter/enhancers can be used to limit expression to the cell types.

### Haemophilia B

An EIAV viral vector (such as those described above) expressing Factor IX (the wild type ORF or a codon optimised ORF) is administered following the methodology in Example 1 and including either intravenous or intra hepatic or intramuscular delivery. A suitable promoter such as CMV or human promoter/enhancers such as PGK is used to express the gene. In addition inducible promoters such as the Tet system can be used to regulate the expression. As an alternative tissue specific promoter/enhancers can be used to limit expression to the cell types.

### EXAMPLE 3

### Cystic Fibrosis

This Example is carried out following the methodology of Example 1. Cystic fibrosis is an hereditary recessive disorder caused by mutation of cystic fibrosis transmembrane conductance regulator (CFTR), a protein that is thought to have a role in ion transport, mucus rheology, inflammation and bacterial adherence. EIAV is used to deliver CFTR by to the amniotic fluid for transduction of lung.

An EIAV viral vector (such as those described above) expressing CFTR (the wild type ORF or a codon optimised ORF) is administered following the methodology in Example 1 and including either intragastrointerstinal delivery intralung or intraamniotic fluid. A suitable promoter such as CMV or human promoter/enhancers such as PGK is used to express the gene. In addition inducible promoters such as the Tet system can be used to regulate the expression. As an alternative tissue specific promoter/enhancers can be used to limit expression to the cell types.

### EXAMPLE 4

### Muscular Dystrophy

This Example is carried out following the methodology of Example 1. Duchenne muscular dystrophy (DMD) is a lethal X-linked recessive disorder. DMD results from genetic deficiency in the level and/or activity of the protein dystrophin in the striated musculature. EIAV is used to deliver of minidystrophin cDNA (corresponding to a mild Becker muscular dystrophy (BMD) phenotype) to the umbilical vein of perinates and/or directly into foetal skeletal muscle.

An EIAV viral vector (such as those described above) expressing dystrophin (the wild type full length open reading frame (ORF) or a truncated wild type ORF or a codon optimised ORF or a truncated codon optimised ORF) is administered following the methodology in Example 1 and including delivery into all muscle groups. A suitable promoter such as CMV or human promoter/enhancers such as PGK is used to express the gene. In addition inducible promoters such as the Tet system can be used to regulate the expression. As an alternative tissue specific promoter/enhancers can be used to limit expression to the cell types.

### EXAMPLE 5

### Ribozyme

This Example is carried out following the methodology of Example 1. A ribozyme which targets a gene on the biosynthetic pathway that generates melanin is delivered using EIAV. This approach facilitates the identification of transgenics.

### EXAMPLE 6

### Use of EIAV for Transgenic Models of Parkinson's

Parkinson's disease (PD) is one of the most common neurodegenerative diseases, affecting almost 2% of the population over 65. The disease is characterised by a movement disorder—parkinsonism—symptoms of which are rigidity, resting tremor and bradykinesia (slowness to initiate and carry out movement). This results from the loss of neurons in the substantia nigra that produce the neurotransmitter dopamine. The causes of PD are largely unknown, although there are a few rare families in which the disease is inherited. In families with autosomal dominant PD two different missense mutations have been mapped in α-synuclein (Polymeropoulos et al 1997; Kruger et al 1998), which is a small phosphoprotein thought to be involved in synaptic vesicle transport. In the case of autosomal recessive juvenile parkinsonism (AR-JP), which develops in adolescence, Kitada et al (1998) showed the gene responsible to be Parkin, an E3 ubiquitin ligase recently proposed to catalyse the ubiquitination of α-synuclein (Shimura et al 2001). It has therefore been suggested that an inability to degrade α-synuclein results in AR-JP and possibly sporadic PD (Haass and Kahle 2001).

The EIAV vector system is used to deliver one or more of the following to mouse spermatogonial stem cells (Nagano et al 2001):
1. ribozyme to Parkin
2. mutant α-synuclein allele
3. ribozyme to tyrosine hydroxylase (enzyme required for dopamine synthesis)

### EXAMPLE 7

### Angiogenesis

The hypoxia inducible factor (HIF) is a transcriptional complex that plays a central role in oxygen homeostasis. The alpha subunit of HIF is targeted for degradation under normoxic conditions by the von Hippel-Lindau ubiquitylation complex that recognizes a hydroxylated proline residue in HIF. Steady state levels of the protein are consequently low and the transcriptional complex cannot form. A family of prolyl-4-hydroxylases have recently been described (Epstein at al 2001) whose enzyme activity is modulated by hypoxia, iron chelation and cobaltous ions, fulfilling the requirements for being oxygen sensors that regulate HIF. Suppression of proly-4-hydroxylase in cultured Drosophila melanogaster cells by RNA interference resulted in elevated expression of a hypoxia-inducible gene under normoxic conditions (Bruick and McKnight 2001).

The EIAV vector system is used to deliver:
1. A ribozyme to prolyl-4-hydroxlase (or VHL). This may lead to constitutive up-regulation of HIF-1 alpha subunits, activation of the HIF complex and overexpression of HIF target genes.
2. Constitutively active HIF-1 (upregulation of HIF in normoxia) or PHD3 (downregulation of HIF in hypoxia).
   to mouse oocytes by injection into the perivitelline space (Chan et al 1998; 2001).

The production and applications of transgenic mouse models in health-related research are well documented. The proposed research will enable the development of models for a broad range of human diseases the generation of which are currently unmet by existing ‘knockout’ methodology.

Advantages over existing technology include the following:

1) Increased efficiency of transgene delivery by lentiviral transduction as compared with non-homologous recombination of injected DNA. Pronuclear injection leads to insertion of large tandem arrays of DNA which are unstable and subject to rearrangements and deletions. Lentiviral transduction generally leads to the stable integration of a limited number of vector copies distributed as discrete cassettes in the chromosomal DNA.

2) Reduction in turnaround time compared to current ‘knock-out’. To produce mice with homozygous gene deletions is a relatively labour-intensive and time-consuming process requiring the cross-breeding of mosaic heterozygotes in which the engineered gene deletion has ‘gone germline’. In contrast, by transducing oocytes prior to fertilisation, every cell will contain the ablation cassette. The need for cross-breeding is by-passed resulting in shorter turnaround times and a substantial decrease in the overall number of animals required.

3) Flexibility of gene product knock-down. As discussed this technology will be of particular value in establishing disease models where deletion of the gene of interest is lethal. It will be advantageous in all studies where ablation of gene expression is desired at particular developmental stages or restricted to specific tissues.

4) HIV vectors have a number of significant disadvantages which may limit their therapeutic application to certain diseases. HIV-1 has the disadvantage of being a human pathogen carrying potentially oncogenic proteins and sequences. There is the risk that introduction of vector particles produced in packaging cells which express HIV gag-pol will introduce these proteins into an individual leading to seroconversion. The present non-primate lentiviral-based vectors used in embodiment of the present invention do not introduce HIV proteins into individuals

### EXAMPLE 8

### Production of Transgenic Avians as Bioreactors for the Production of Proteins

An EIAV vector encoding for bacterial β-galactosidase, initially 10 microlitres of pONY8Z 5′cppt, is injected directly below the blastoderm stage embryo in new-laid eggs using published technology such as is described in U.S. Pat. No. 5,258,307 or earlier stage embryos using techniques such as those described in WO 90/13626. An inert dye is used for blastoderm stage injections to ensure accurate delivery of the vector. Transduction efficiency is analysed by harvesting embryos at different stages of development during incubation and after hatching. The embryos are then sectioned and stained for β-galactosidase activity to identify which organs are transduced and whether germ cells in the embryonic gonad were transduced. Samples of tissues such as blood and CAM are taken from the embryos and the percentage of cells transduced will be assessed using quantitative PCR. Some of the male embryos are grown to sexual maturity and semen samples will be taken. The likelihood of such birds passing the transgene on to any offspring is assessed using quantitative PCR. The semen is then used to inseminate hens and the embryos are harvested and assessed by quantitative PCR to determine the percentage of transgenic offspring. In addition the level of β-galactosidase expression from the vector in the G₁ population is assessed by staining using X-Gal.

### EXAMPLE 9

### Vectors for Use in RNA Interference (RNAi)

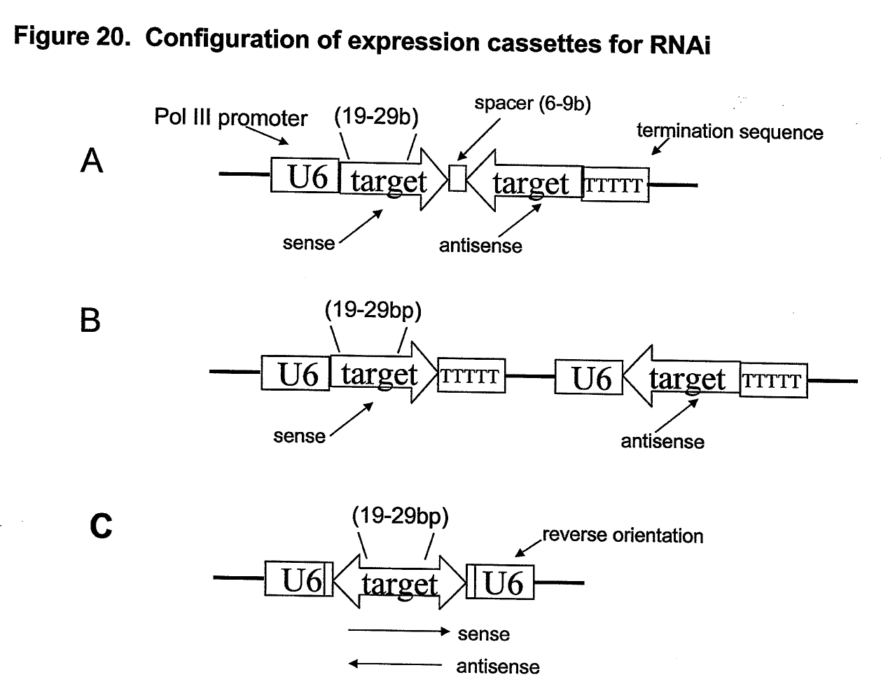

FIG. 20 illustrates a number of expression cassettes for RNAi which can be used in lentiviruses, for example EIAV) to express siRNA in transgenic cells and animals. In each of these examples, an RNA polymerase III promoter (U6) has been utilised. RNA polymerase III makes a variety of very small, stable RNAs including the small 5S ribosomal RNA and the transfer RNAs. Effective RNAi is mediated by either expression of a short hairpin from a single U6 promoter (FIG. 20A) or incorporating two U6 promoters, one expressing a region of sense and the other the reverse complement of the same sequence of the target (FIG. 20B). In the embodiment shown in FIG. 20C, two opposing promoters are used to transcribe the sense and antisense regions of the target from the forward and complementary strands of the expression cassette.

### EXAMPLE 10

### Aptazymes for Regulating Production of Short RNAs

The use of viral vectors, e.g. lentiviral vectors for generating transgenics to deliver siRNAs which target a gene product with an important or essential function may result in death of the transgenic animal during development. The ability to regulate transcription of the siRNAs, thereby allowing the silencing effect to be modulated, would be greatly desirable. In this example, we describe the use of an aptamer/ribozyme hybrid (‘aptazyme’) for regulating the production of functional siRNAs.

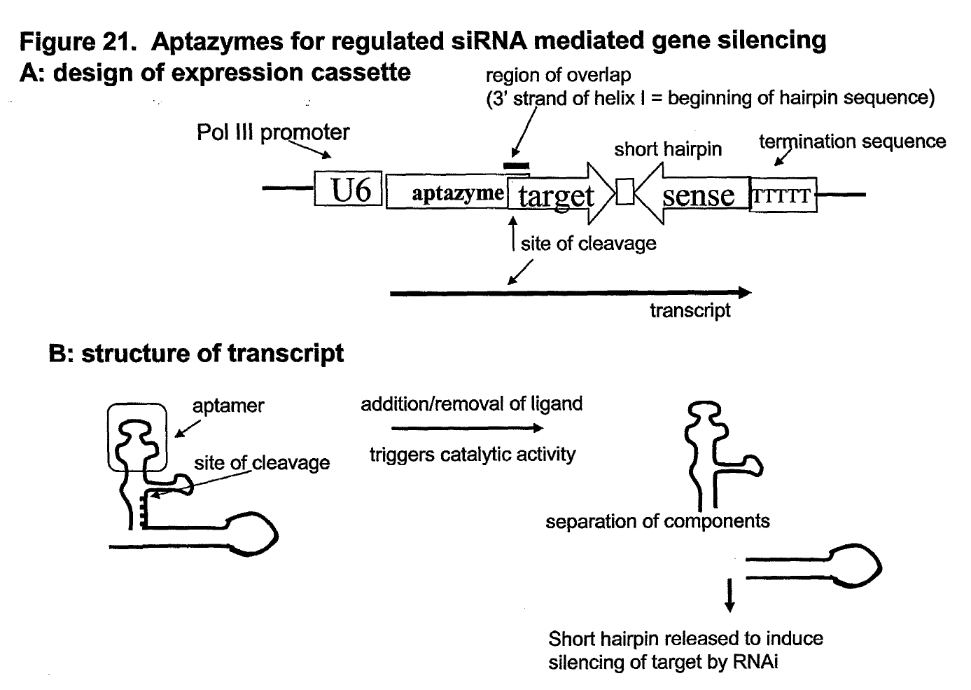

Aptamers are nucleic acid molecules which form structures which are able to bind a number of ligands including proteins and drug molecules. By replacing one helix of a hammerhead ribozyme with an aptamer it has been possible to create a catalytic RNA which is able to cleave a substrate (which may be itself) as the result of conformational change induced by the presence or absence of a ligand. FIG. 21A illustrates the design of an expression cassette which may be used in vectors of the invention and methods of the invention. In this cassette, an aptazyme is added 5′ of siRNA encoded by a short hairpin. This allows the regulated induction (or inhibition) of self-cleavage of the transcript separating the hairpin from the aptazyme structure and hence activating silencing. As illustrated in FIG. 21B, addition/removal of a ligand triggers catalytic activity, cleaving the transcript and allowing release of the short hairpin to induce silencing of a target sequence.

### EXAMPLE 11

### Hypoxically Induced Silencing of VEGF by siRNAs

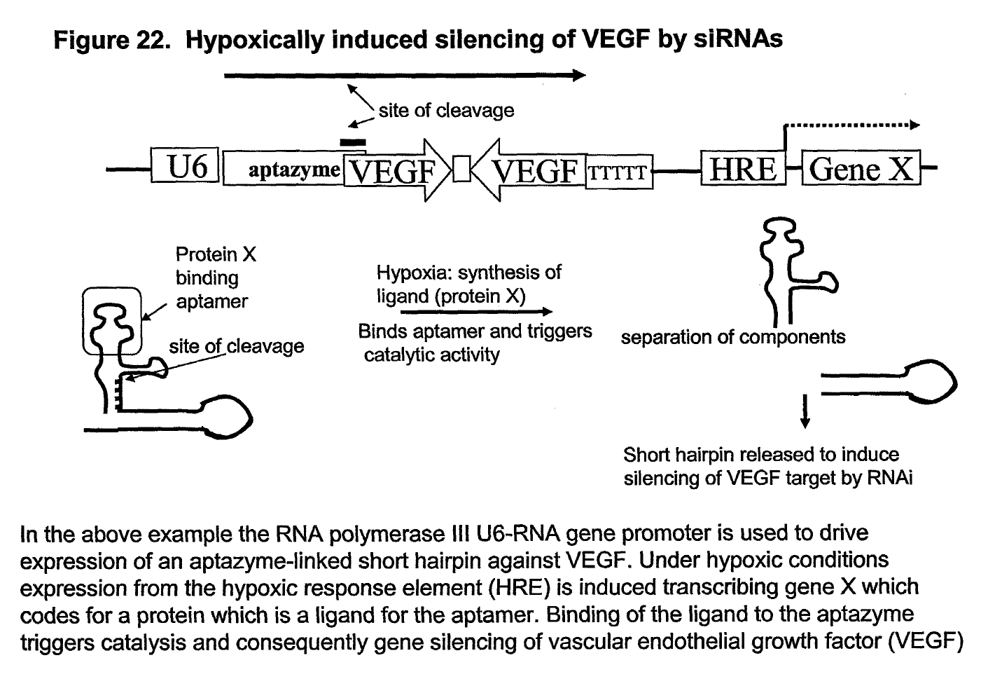

In this example, we describe the use of an ‘aptazyme’ for regulating the production of functional siRNAs, in which the protein ligand specific for the aptamer is expressed from the same vector. A vector is constructed as illustrated in FIG. 22. The RNA polymerase III U6snRNA gene promoter is used to drive expression of an aptazyme-linked short hairpin against VEGF. Under hypoxic conditions expression from the hypoxic response element (HRE) is induced transcribing gene X which codes for a protein X which is a ligand for the aptamer. Binding of the ligand to the aptazyme triggers catalysis, release of the short hairpin and consequently gene silencing of vascular endothelial growth factor (VEGF)

Thus VEGF is specifically down-regulated in hypoxia which may be therapeutically beneficial in a number of diseases including proliferative diabetic retinopathy.

In an alternative embodiment, the ligand for the aptamer may be VEGF itself.

### EXAMPLE 12

### Use of RNA Polymerase II Promoters for Transcribing siRNA Precursors

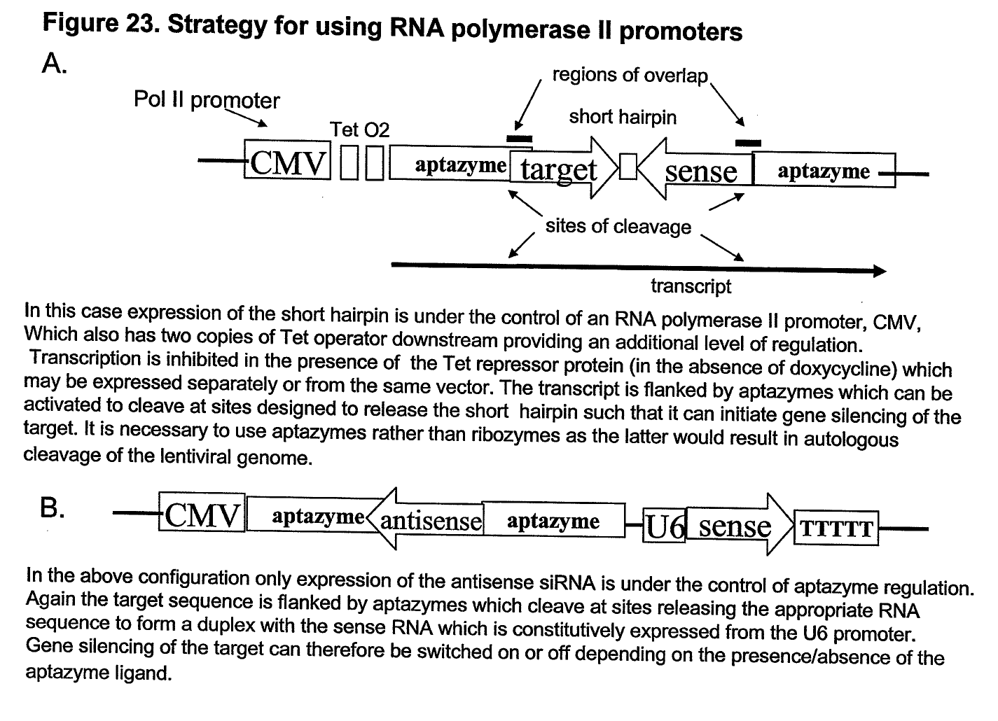

In this example, we describe the transcription of siRNA precursors under the control of RNA polymerase II promoters. This achieved by flanking the short hairpin (FIG. 23A), or siRNA sequence (FIG. 23B) with sequence which codes for, or is a target of, a catalytic RNA such as an aptazyme. Cleavage of the flanking sequences releases the siRNA or short hairpin from the precursor.

In the expression cassette shown in FIG. 23A, expression of the short hairpin is under the control of an RNA polymerase II promoter, CMV. Two copies of Tet operator downstream provide an additional level of regulation. Transcription is inhibited in the presence of the Tet repressor protein (in the absence of doxycycline) which may be expressed separately or from the same vector. The transcript is flanked by aptazymes which can be activated to cleave at sites designed to release the short hairpin such that it can initiate gene silencing of the target. In this particular embodiment, it is necessary to use aptazymes rather than ribozymes as the latter would result in autologous cleavage of the lentiviral genome.

In the expression cassette shown in FIG. 23B, only expression of the antisense siRNA is under the control of aptazyme regulation. Again the target sequence is flanked by aptazymes which cleave at sites releasing the appropriate RNA sequence to form a duplex with the sense RNA which is constitutively expressed from the U6 promoter. Gene silencing of the target can therefore be switched on or off depending on the presence/absence of the aptazyme ligand.

### EXAMPLE 13

### Use of Vectors Comprising Aptazyme Sequences for Regulating Expression of Transgenes

Aptazymes may be used for post-transcriptional regulation of any nucleic acid sequence including genes. The aptazyme is activated (or inhibited) by the addition/removal of the appropriate ligand inducing cleavage of the transcript, either removing part of the transcript, for example the codon for the initiator methionine, or a UTR preventing capping and/or polyadenylation of the transcript. This provides a means of shutting off synthesis of a gene product, for example a therapeutic gene such as Factor IX, if levels are too high. Expression of the transgene can be engineered to be self-regulating in this way.

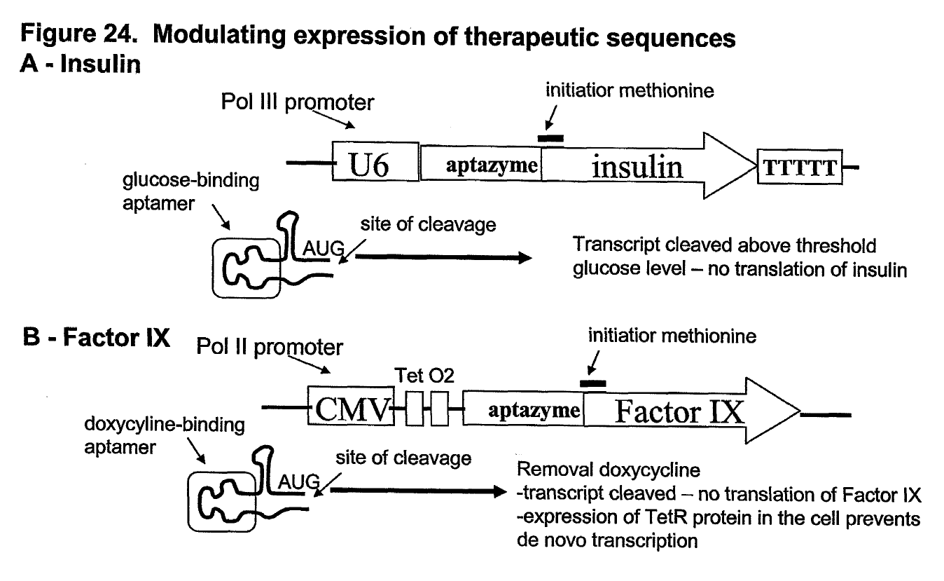

FIG. 24A illustrates a construct for use in modulation of expression of insulin. The aptazyme whose activity is modulated by glucose binding is designed such that high level expression of insulin occurs only when blood glucose levels are high. If glucose fall below a threshold level than the aptazyme is active and the insulin transgene transcript destroyed.

FIG. 24B illustrates a construct for use in modulation of expression of Factor IX. In this construct, an aptazyme which is regulated by doxycycline is used to regulate the expression of transgenes/short RNAs both in vitro and in vivo by the administration of doxycycline. In this construct, Tet operator sequences are inserted downstream of the promoter. Transcription is repressed in the presence of the Tet repressor protein (which may be expressed from the same vector) when doxycycline is removed, thereby preventing de novo transcription. As the aptazyme is active in the absence of doxycycline any existing transcripts will be cleaved and degraded.

The T-Rex™ (InVitrogen) system could optionally be incorporated in such a strategy to add an additional level of control.

### EXAMPLE 14

### Use of Vectors Comprising Aptazyme Sequences for Regulating Expression of Transgenes—Measures to Prevent Self-Cleavage of Vector Genome RNA

Although production of viral vectors of the invention should be carried out under conditions which should minimise activity of the aptazyme, and hence unwanted destruction of the vector genome by self-cleavage, a preferred measure is to physically separate the aptazyme (or ribozyme) by configuring the vector as a split intron vector (Ismail et al 2000). This ensures that the full sequence of the ribozyme is only present in the transcript encoded by the provirus and not in the RNA genome present in the vector particle.

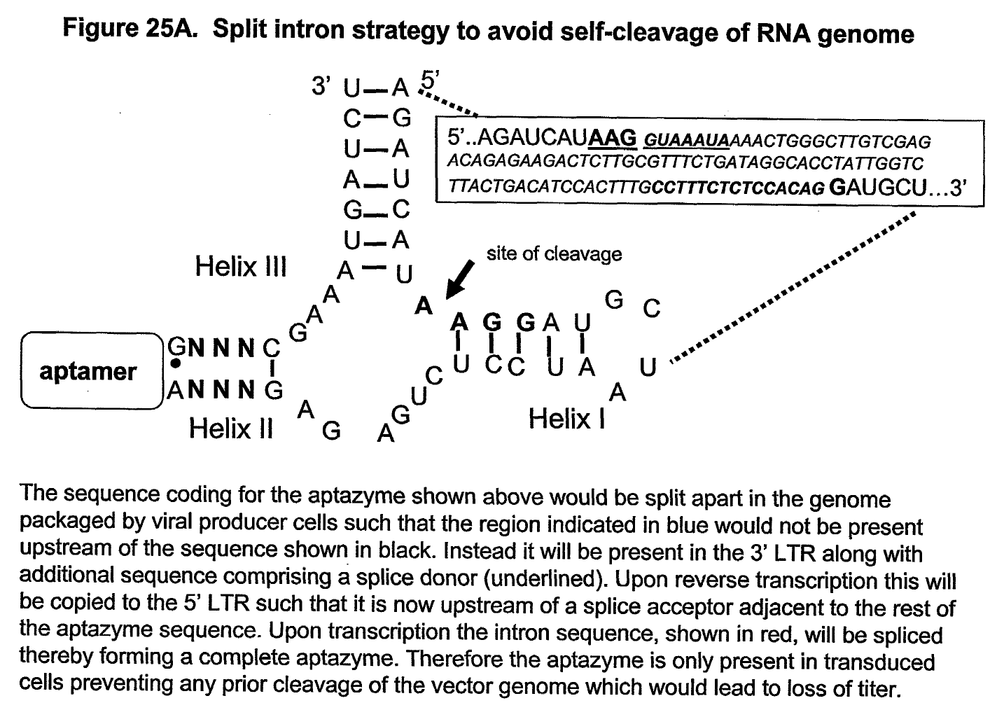

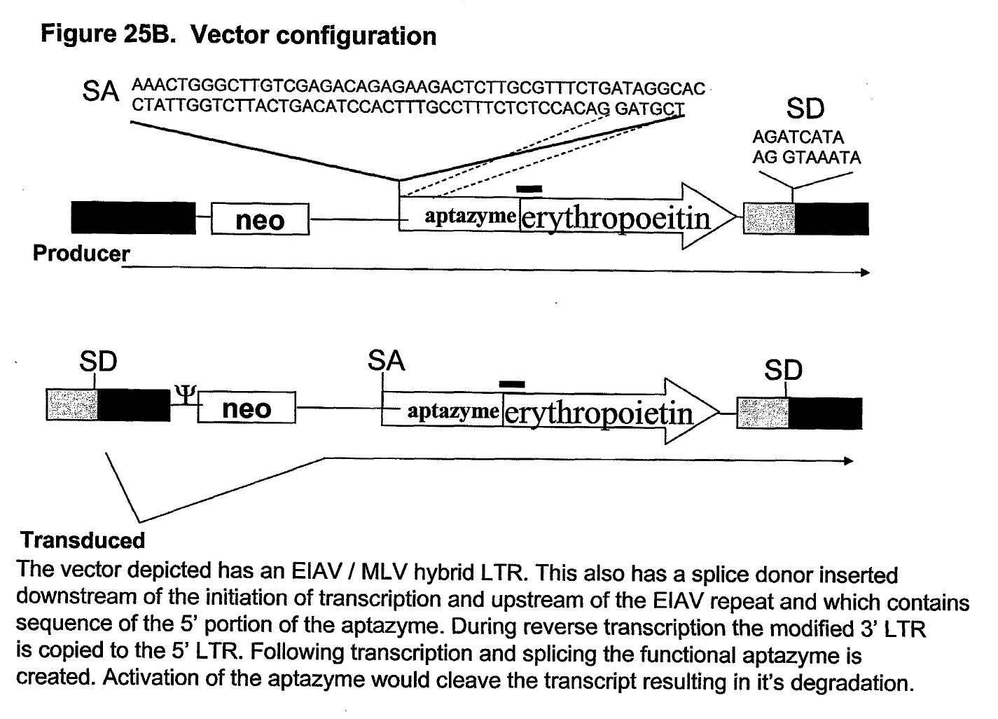

FIG. 25A illustrates the split intron strategy with FIG. 28B illustrating suitable vector of this aspect of the invention. During the process of reverse transcription a splice donor, and some of the 5′ sequence of the ribozyme, is copied to the 5′ viral LTR such that the ribozyme is created only following splicing of the transcribed provirus. Using the specific example shown in FIG. 25A, the sequence coding for the aptazyme would be split apart in the genome packaged by viral producer cells such that the region indicated in blue (AGAUCAU) would not be present upstream of the sequence shown in black (GAUGCU). Instead it will be present in the 3′ LTR along with additional sequence comprising a splice donor (underlined). Upon reverse transcription this will be copied to the 5′ LTR such that it is now upstream of a splice acceptor adjacent to the rest of the aptazyme sequence. Upon transcription the intron sequence, underlined:

(GUAAAUAAAACTGGGCTTGTCGAGACAGAGAAGACTCTTGCGTTTCTGAT AGGCACCTATTGGTCTTACTGACATCCACTTTGCCTTTCTCTCCACAG), will be spliced thereby forming a complete aptazyme. Therefore the aptazyme is only present in transduced cells preventing any prior cleavage of the vector genome which would lead to loss of titre.

FIG. 25B illustrates a split intron vector which may be used in this aspect of the invention. The vector has an EIAV/MLV hybrid LTR. This also has a splice donor inserted downstream of the initiation of transcription and upstream of the EIAV repeat and which contains sequence of the 5′ portion of the aptazyme. During reverse transcription the modified 3′ LTR is copied to the 5′ LTR. Following transcription and splicing the functional aptazyme is created. Activation of the aptazyme cleaves the transcript resulting in its degradation.

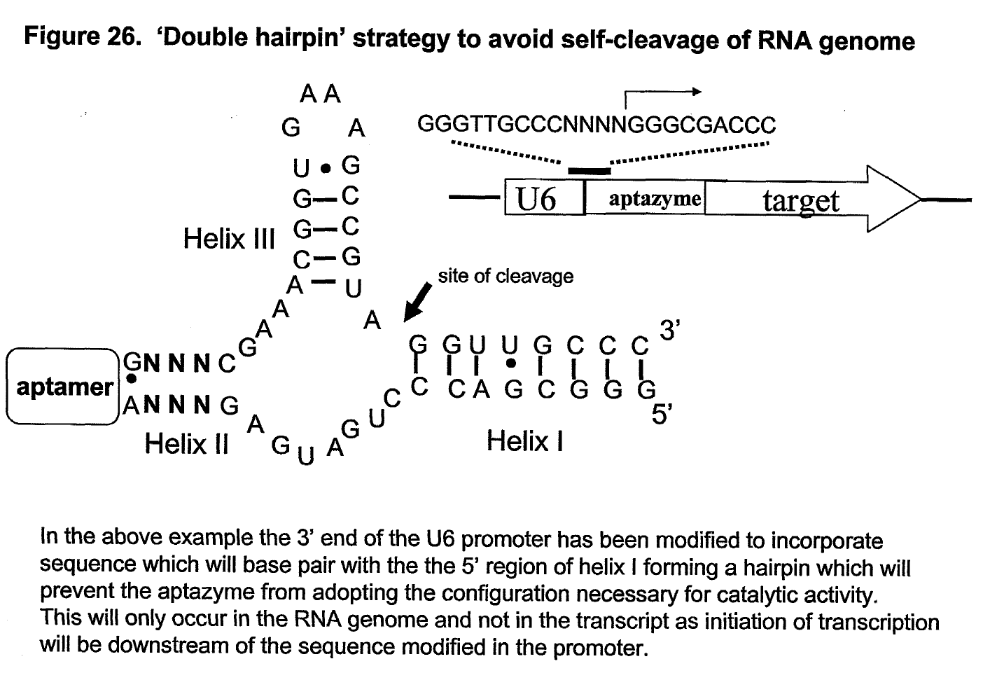

An additional means of preventing formation of a potentially active aptazyme within the viral RNA genome is to include sequence at the 3′ end of the promoter which is able to base-pair with a part of the aptazyme reducing the possibility of it adopting the correct configuration. This is illustrated in FIG. 26. As shown, the 3′ end of the U6 promoter has been modified to incorporate sequence which will base pair with the 5′ region of helix I forming a hairpin which will prevent the aptazyme from adopting the configuration necessary for catalytic activity. This will only occur in the RNA genome and not in the transcript as initiation of transcription will be downstream of the sequence modified in the promoter. This would not interfere with the tertiary structure of the transcribed provirus as promoter sequences would not be present.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

### REFERENCES

Nagano M, Brinster C J, Orwig K E, Ryu B Y, Avarbock M R, Brinster R L.
Proc Natl Acad Sci USA. 2001 Nov. 6; 98(23):13090-5.
Chan A W, Homan E J, Ballou L U, Burns J C, Bremel R D.
Proc Natl Acad Sci USA. 1998 Nov. 24; 95(24):14028-33.
Chan A W, Chong K Y, Martinovich C, Simerly C, Schatten G.
Science. 2001 Jan. 12; 291(5502):226
Vilotte J L, L'Huillier P, Mercier J C.
J Mammary Gland Biol Neoplasia 1998 July; 3(3):351-362
Polymeropoulos et al (1997) Science 276(5321):2045-7
Kruger et al (1998) Nat Genet. 18(2):106-8
Kitada et al (1998) Nature 392(6676):605-8
Shimura et al (2001) Science 293(5528):239-9
Haas and Kahle (2001) Science 293(5528):224-5
Epstein et al (2001) Cell 107(1):43-54
Bruick and McKnight (2001) Science 294(5545):1337-40
Soukup and Breaker (1999) Structure Fold Des. July 15; 7(7):783-91
Van Deutekom et al (2001) Hum Mol Genet July 15; 10(15):1547-54
Vickers et al (2001) Nucleic Acids Res. March 15; 29(6):1293-9
Lagos-Quintana et al (2001) Science October 26; 294 (5543):853-8
Ohkawa et al (2000) Hum Gene Ther. March 1; 11(4):577-85
Stetor S R, Rausch J W, Guo M J, Burnham J P, Boone L R, Waring M J, Le Grice S F.
Characterization of (+) strand initiation and termination sequences located at the center of the equine infectious anemia virus genome. Biochemistry 1999 Mar. 23; 38(12):3656-67
Donello J E, Loeb J E, Hope T J.
Woodchuck hepatitis virus contains a tripartite posttranscriptional regulatory element. J Virol 1998 June; 72(6):5085-92
Mitrophanous K, Yoon S, Rohll J, Patil D, Wilkes F, Kim V, Kingsman S, Kingsman A, Mazarakis N.
Stable gene transfer to the nervous system using a non-primate lentiviral vector. Gene Ther 1999 November; 6(11):1808-18

1-84. (canceled)

85. A method of producing a transgenic avian cell comprising introducing into an avian cell a non-primate lentiviral expression vector comprising a nucleotide of interest (NOI).

86. The method according to claim 85, wherein the NOI encodes and expresses a therapeutic protein or an aptazyme, or generates an antisense oligonucleotide, a ribozyme, an siRNA, a short hairpin RNA, a micro-RNA or a group 1 intron.

87. The method according to claim 85, wherein the non-primate lentiviral expression vector is derived from EIAV, FIV, BIV, CAEV or MVV.

88. The method according to claim 85, wherein the expression vector is introduced into the avian cell in vivo or ex vivo.

89. The method according to claim 85, wherein the avian cell is capable of giving rise to a germ line change.

90. The method according to claim 89, wherein the avian cell is involved in gametogenesis.

91. The method according to claim 89, wherein the avian cell is an oocyte, an oviduct cell, an ovarian cell, an ovum, an oogonium, a zygote, an embryonic stem (ES) cell, a blastocyte, a spermatocyte, a spermatid, a spermatozoa, or a spermatogonia.

92. The method according to claim 85, wherein the lentiviral expression vector is pseudotyped.

93. The method according to claim 85, wherein the lentiviral expression vector does not contain any functional accessory genes.

94. The method according to claim 85, wherein the NOI is operably linked to a constitutive, tissue-specific or an inducible promoter.

95. A transgenic avian cell comprising a non-primate lentiviral expression vector comprising a nucleotide of interest (NOI).

96. A transgenic avian organism which is generated from the transgenic avian cell of claim 95.

97. A transgenic egg derived from the transgenic avian organism according to claim 96.
